(19) United States
(12) Patent Application Publication
(10) Pub. No.: US 2026/0213005 A1
(43) Pub. Date: Jul. 23, 2026

(54) SYSTEMS AND METHODS FOR DELIVERING USER SPECIFIC MESSAGES

(71) Applicant: Datum Point Labs, Inc., Jackson, WY (US)

(72) Inventors: Patrick Nunally, San Diego, CA (US); Ruy Carrasco, Austin, TX (US)

(21) Appl. No.: 19/563,740

(22) Filed: Mar. 11, 2026

Related U.S. Application Data
(63) Continuation-in-part of application No. 18/623,834, filed on Apr. 1, 2024, now Pat. No. 12,581,163.
(60) Provisional application No. 63/457,696, filed on Apr. 6, 2023.

(51) Int. Cl. G16H 40/20

(52) U.S. Cl. CPC G16H 40/20

(57) ABSTRACT

Embodiments described herein provide systems and methods for delivering user specific messages. A system receives a media feed including at least one of an audio feed or a video feed. The system determines, based on the media feed, a categorization of the media feed. The system determines, based on a set of information, a replacement media. The system overrides the media feed with the replacement media based on the categorization.

### CROSS REFERENCE

The instant application is a Continuation-in-Part of U.S. patent application Ser. No. 18/863,834, filed Apr. 1, 2024, which claims priority to U.S. Provisional Application No. 63/457,696, filed Apr. 6, 2023, both of which are incorporated by reference herein in their entirety.

### BACKGROUND

Existing medical communication systems (e.g. EHR, EMR, practice management software; Clinical Communication & Collaboration (CC & C), patient engagement & messaging; healthcare CRM)) typically rely on standardized guidelines, generalized treatment protocols, and broad population-level data when delivering information to physicians. While patients may be divided into large classes, for example by diagnosis, acuity & setting, service lines, age group, or geographic region, such categorization often fails to reflect the unique clinical profile, history, preferences, and risk factors of an individual patient. This is unlikely to be well-suited to an individual user's needs.

Further, information provided to physicians is often static, delayed, or controlled by centralized content publishers such as guideline committees, insurers, or institutional systems. Therefore, there is a need for improved systems and methods for delivering patient-specific medical information and recommendations to physicians at the point of care.

### BRIEF DESCRIPTION OF THE DRAWINGS

FIG. 1 illustrates a framework for user specific messaging, according to some embodiments.

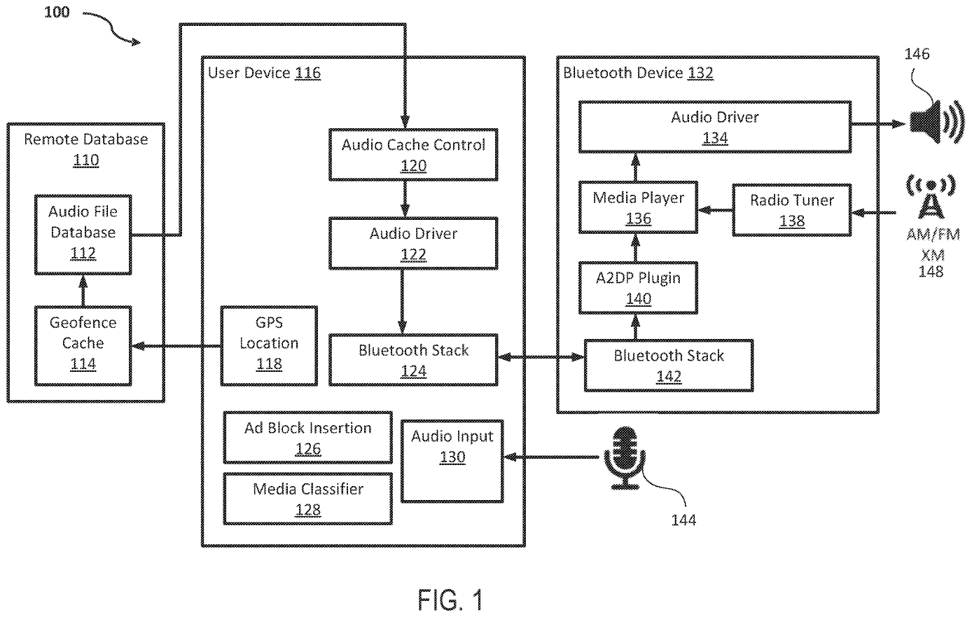

FIG. 2 illustrates a framework for user specific messaging, according to some embodiments.

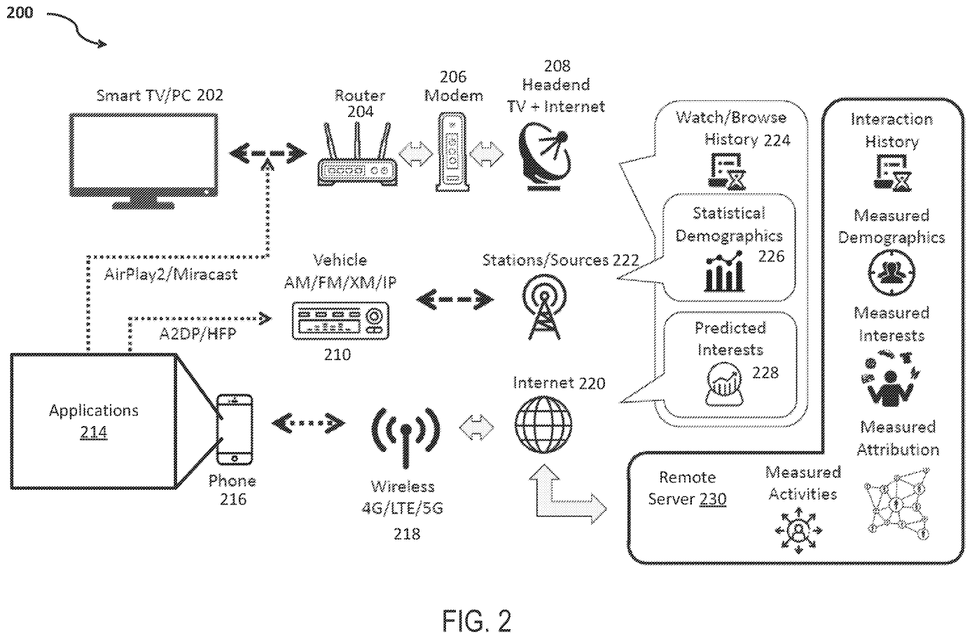

FIG. 3 illustrates a framework for user specific messaging for television, according to some embodiments.

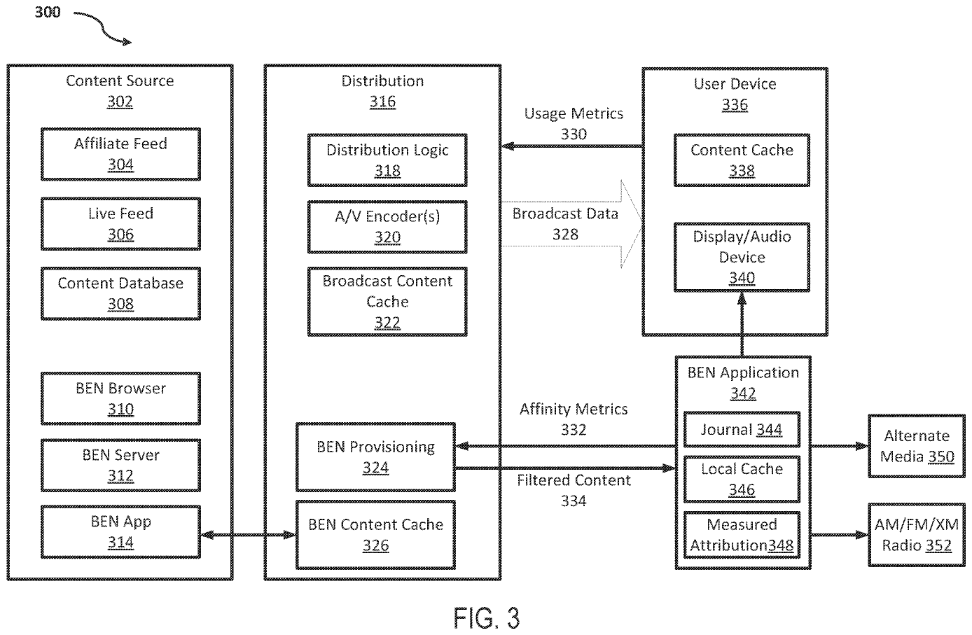

FIG. 4 is a simplified diagram illustrating a computing device implementing the framework described herein, according to some embodiments.

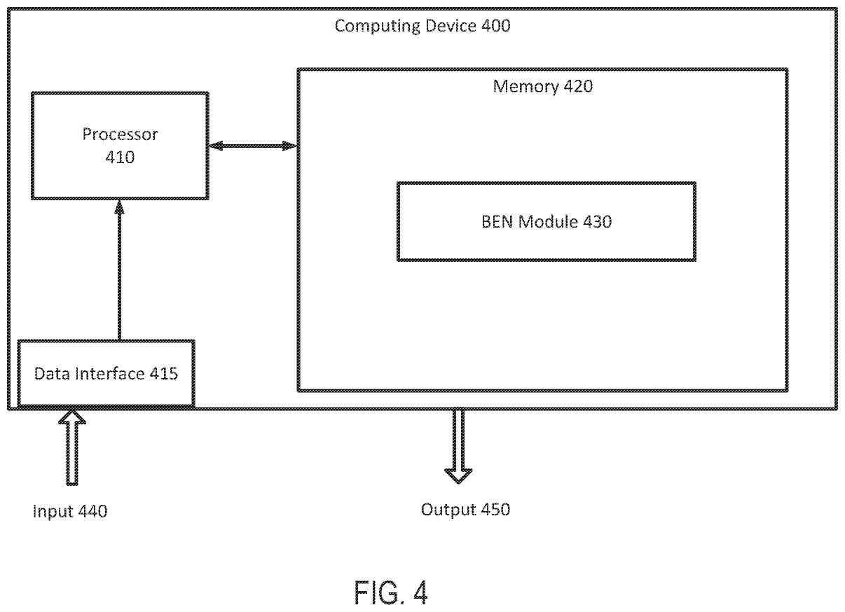

FIG. 5 is a simplified diagram illustrating a neural network structure, according to some embodiments.

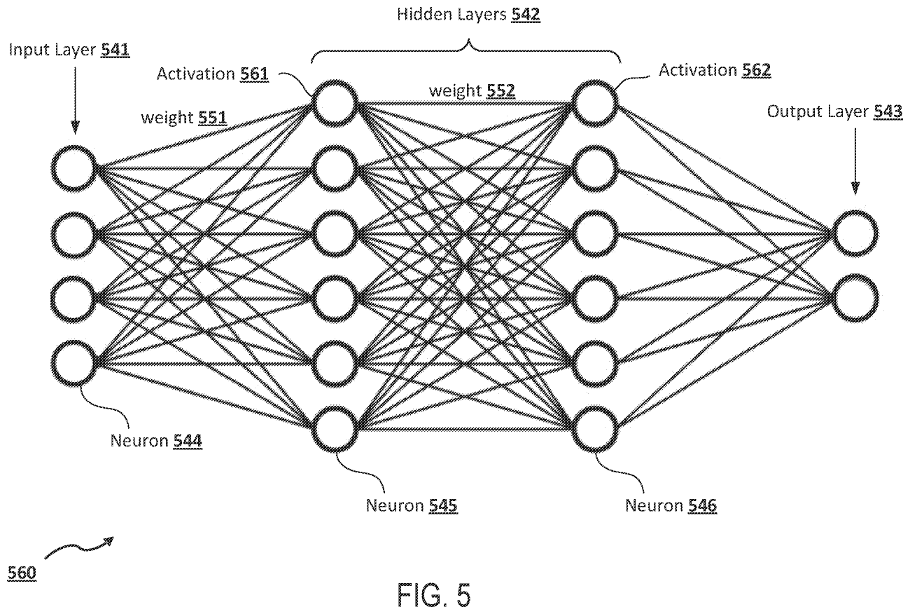

FIG. 6 is a simplified block diagram of a networked system suitable for implementing the framework described herein.

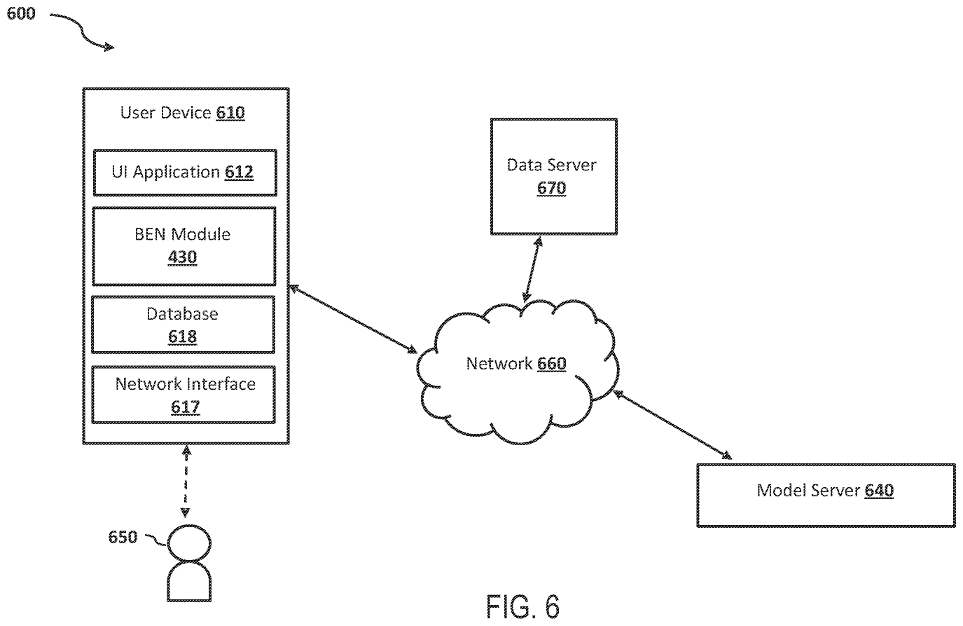

FIG. 7 is an example logic flow diagram, according to some embodiments.

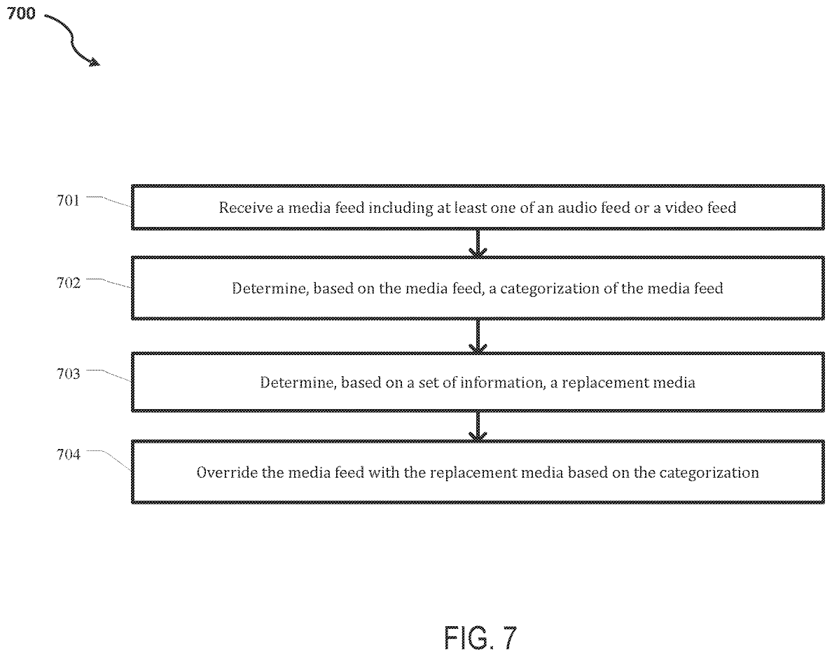

FIGS. 8A-8B are exemplary devices with digital avatar interfaces, according to some embodiments.

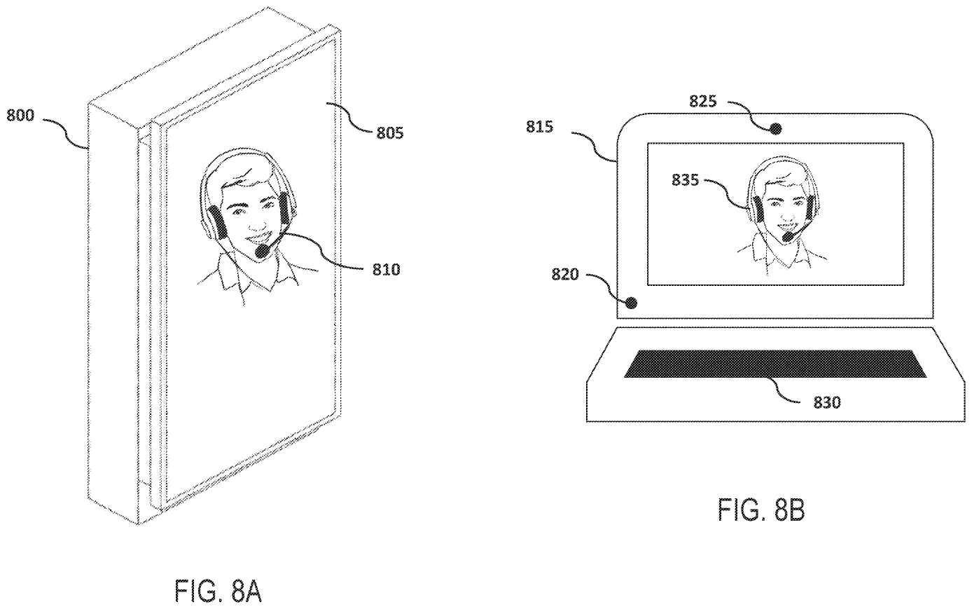

### DETAILED DESCRIPTION

Healthcare delivery increasingly relies on electronic health records, diagnostic systems, clinical documentation tools, patient engagement & messaging and real-time data streams to support physician decision-making. However, existing clinical decision support and medical communication systems typically deliver static, population-level guidance that is poorly adapted to the unique and evolving clinical context of an individual patient. Such systems often rely on fixed rules, generalized alerts, or guideline-driven prompts that do not adequately account for patient-specific history, concurrent conditions, care setting, allergies (food, medication), workflow timing, or physician interaction patterns. As a result, clinically relevant information may be delivered too late, in an inappropriate format, or in a manner that disrupts clinical workflow, contributing to alert fatigue and reduced clinical utility.

Embodiments described herein relate to a multi-layer adaptive clinical workflow engine configured to deliver patient-specific medical information to a caregiver at the point of care. In contrast to conventional alerting systems, the disclosed framework operates as an adaptive orchestration layer that continuously ingests clinical information, evaluates relevance across multiple factors, calibrates presentation behavior based on observed outcomes and clinician interaction, and injects guidance into clinical workflow in a non-disruptive manner. The system is designed to operate dynamically within active clinical encounters and to evolve over time as patient state, physician behavior, and clinical evidence change.

In some embodiments, systems and methods for patient-specific medical messaging delivered to physicians and other health professionals are included. Embodiments herein take principal advantage of the fact that physicians interact continuously with patient data through electronic health records (EHRs), diagnostic imaging, laboratory systems, genomic data, proteome & interactome insight, metabolomic data, wearable devices, and clinical documentation, and that clinical communication systems must fundamentally convey content to physicians at the point of care.

In some embodiments, the system includes a clinical feed ingestion layer configured to receive and normalize heterogeneous clinical data associated with an active patient context. The clinical feed may include, by way of example, structured and unstructured electronic health record data, laboratory results, imaging reports, medication orders, device or wearable data, dictated or transcribed clinical notes, and other observable patient indicators. The ingestion layer may continuously update the active patient context as new information becomes available, thereby enabling downstream components to operate on current and clinically meaningful data rather than static snapshots.

In some embodiments, the system further includes a multi-factor relevance scoring layer configured to evaluate candidate medical information, recommendations, or replacement media with respect to the active patient context. Relevance scoring may incorporate multiple weighted factors, including patient-specific clinical attributes, acuity or risk indicators, temporal proximity to workflow events, care setting, physician role or specialty, and historical interaction patterns. The relevance scoring layer enables the system to prioritize which insights are likely to provide the greatest clinical value for a given patient and moment in workflow, rather than treating all alerts or guidance as equivalent.

In some embodiments, the system further includes an adaptive calibration layer configured to adjust relevance thresholds, weighting factors, presentation timing, and insertion modality based on observed physician interactions and downstream clinical outcomes. The calibration layer may operate continuously or periodically and may be informed by acceptance, modification, deferral, override, and feedback events, as well as subsequent changes in patient state. This adaptive behavior allows the system to become increasingly personalized over time, reducing unnecessary interruptions while preserving or enhancing delivery of high-value guidance.

In some embodiments, the system further includes a learning and feedback layer configured to capture physician interaction data, outcome indicators, and workflow context, and to use such information to refine patient-specific modeling. The learning layer may link interaction events to corresponding patient contexts and recommendations, enabling the system to identify patterns of clinical utility, workflow friction, or alert fatigue. The learning layer may operate subject to guardrails that limit the rate and magnitude of adaptive changes, preserve baseline configurations, and ensure that clinician judgment remains authoritative.

In some embodiments, the system further includes a non-disruptive workflow injection layer configured to insert selected medical information into one or more points in clinical workflow without interrupting clinical reasoning. Workflow injection may include inserting content during chart open, diagnosis entry, medication ordering, organ function changes (e.g. decreased GFR—renal function), Black Box warning changes, allergy development, chain supply changes (e.g. supply of medication or equipment changes), documentation, or review transitions, and may include inline displays, contextual summaries, or pre-populated documentation elements rather than modal alerts. By separating relevance determination from presentation mechanics, the system enables clinically meaningful guidance to be delivered at the right time, in the right format, and through the right channel.

Collectively, the disclosed embodiments provide a unified adaptive clinical workflow engine that integrates clinical feed ingestion, relevance scoring, adaptive calibration, learning feedback, and non-disruptive workflow insertion. This architecture enables patient-specific medical information to be delivered in a manner that aligns with real-world clinical practice, evolves over time, and supports improved caregiver efficiency and patient outcomes without increasing cognitive burden.

In some embodiments described herein, the term clinical feed refers to a feed of clinical information associated with an active patient context. In some embodiments, the clinical feed includes structured and unstructured clinical data and is received by a system to enable determining a categorization, determining replacement media, and overriding based on the categorization, as described herein. In some embodiments, the clinical feed includes structured and unstructured clinical data. In some embodiments, structured clinical data includes at least one of laboratory results, vital signs, medication lists, problem lists, allergy lists, orders, and other structured entries that are associated with the active patient context. In some embodiments, unstructured clinical data includes at least one of clinical documentation, notes, imaging reports, and other narrative or report content that is associated with the active patient context. In some embodiments, the active patient context corresponds to an active encounter. In some embodiments, the active patient context corresponds to a clinical workflow in which clinical information is received, updated, and reviewed while a physician is providing care at the point of care. In some embodiments, the clinical feed is received from an electronic health record system. In some embodiments, the clinical feed is received from a clinical data server that is in communication with an electronic health record system. In some embodiments, the clinical feed is received from one or more clinical systems, including diagnostic imaging systems, laboratory systems, wearable devices, and clinical documentation systems, that provide clinical information associated with the active patient context. In some embodiments, the clinical feed also includes more general medical information that is not limited to a single patient. In some embodiments, such more general medical information includes treatment strategies. In some embodiments, such more general medical information includes medical journals. In some embodiments, such more general medical information includes clinical guidelines or other reference information that may be used in determining a replacement media for presentation to a physician at the point of care. In some embodiments, the clinical feed also includes operational medical information associated with availability of medicines and treatments including the regulatory approval of new products, treatments, or pharmaceuticals. In some embodiments, the operational medical information includes stock levels of pharmaceuticals. In some embodiments, the operational medical information includes locations of medicines. In some embodiments, such stock levels or locations correspond to a hospital system, an outpatient clinic, a pharmacy, a dispensing cabinet, a storage location, or other supply location that is relevant to care delivery.

In some embodiments described herein, the term replacement media refers to content selected or generated for presentation in place of, or in augmentation of, at least a portion of the clinical feed within a physician workflow. In some embodiments, the replacement media includes patient-specific medical information, recommendations, summaries, diagnostic support, treatment options, documentation suggestions, order sets, or other contextualized guidance determined to be more clinically relevant than generic alerts or background recommendations for an active patient context.

In some embodiments, the operational medical information further includes device availability information and care team capacity information associated with a hospital system, outpatient clinic, telehealth environment, home-based care environment, or other care setting relevant to the active patient context. In some embodiments, device availability information identifies whether a diagnostic, therapeutic, monitoring, or imaging device relevant to a contemplated recommendation is currently available, accessible, or scheduled for use. In some embodiments, care team capacity information identifies availability, workload, scheduling constraints, equipment availability, operating room availability, or role-specific capacity of physicians, nurses, pharmacists, specialists, technicians, or other personnel relevant to execution of a recommended action.

In some embodiments, the clinical feed is received by a system to enable determining a categorization, determining replacement media, and overriding based on the categorization, as described herein. In some embodiments, the clinical feed is received directly by the system from a source that provides the clinical information. In some embodiments, the clinical feed is received indirectly by the system from another component that aggregates, formats, or presents the clinical information. In some embodiments, the clinical feed is received as a sequence of updates associated with the active patient context. In some embodiments, the clinical feed includes clinical information that is received in real time. In some embodiments, the clinical feed includes clinical information that is received periodically. In some embodiments, the clinical feed includes clinical information that is received in response to a clinical workflow transition, including chart open, diagnosis entry, or medication ordering. In some embodiments, the clinical feed includes information sufficient for determining, based on the clinical feed, a categorization of the clinical feed. In some embodiments, the categorization indicates a patient state or a clinical context within the active patient context. In some embodiments, the categorization indicates an emerging risk state. In some embodiments, the categorization indicates an early sepsis risk, a medication interaction risk, or a deteriorating heart failure indicator. In some embodiments, the clinical feed includes information usable to determine, based on a set of information, a replacement media. In some embodiments, the set of information includes patient-specific clinical history, genomic or biomarker information, real-time lab values, medication interactions, social determinants of health, behavioral and adherence data, time-sensitive clinical guidelines, and physician specialty and treatment style.

In some embodiments, overriding based on the categorization includes inserting contextualized guidance into a physician workflow. In some embodiments, overriding includes presenting patient-specific medical information and recommendations to a physician at the point of care. In some embodiments, the clinical feed includes, or is associated with, patient location information and time of day information. In some embodiments, the clinical feed includes, or is associated with, historical information associated with the active patient context. In some embodiments, the clinical feed is received by the system in a form that is suitable for classification. In some embodiments, the clinical feed is received as a full representation of clinical information. In some embodiments, the clinical feed is received as a reduced representation of clinical information.

Rather than relying solely on pre-coded rules or static guideline triggers, embodiments herein evaluate structured and unstructured clinical data to classify patient state and context in real time.

In some embodiments, these systems mimic how a physician synthesizes patient information, integrating symptoms, labs, imaging, medication history, behavioral indicators, and risk patterns, to generate relevant, timely clinical insights. In some embodiments, the synthesis includes evaluating the symptoms in view of laboratory results and imaging results to determine whether a set of findings is consistent with an emerging clinical state. In some embodiments, the synthesis includes evaluating medication history together with current lab values and documented symptoms to identify a medication interaction risk or other adverse pattern. In some embodiments, the synthesis includes evaluating behavioral indicators and risk patterns together with time-varying lab trends to identify elevated risk within the boundaries of an active encounter, including when a patient is not explicitly labeled as high risk. In some embodiments, the synthesis includes determining, based on the clinical feed, a categorization of the clinical feed that reflects patient state and context, and determining, based on a set of information, replacement media that provides patient-specific medical information and recommendations delivered to a physician at the point of care. In some embodiments, the relevant, timely clinical insights include contextualized guidance that is inserted into a physician workflow without disrupting higher-priority clinical tasks.

Upon classification of a patient's condition or emerging risk state, the system may insert contextualized, patient-specific guidance into the physician's workflow without disrupting higher-priority clinical tasks. In some embodiments, the system receives a clinical feed associated with an active patient context and determines, based on the clinical feed, a categorization of the clinical feed that indicates the patient's condition or emerging risk state. In some embodiments, the system determines, based on a set of information, a replacement media that includes patient-specific medical information and recommendations delivered to a physician at the point of care, and the system overrides the clinical feed with the replacement media based on the categorization. In some embodiments, overriding based on the categorization includes inserting contextualized guidance into the physician's workflow within an electronic health record user interface, within a clinical decision-support dashboard, or within a mobile clinical device interface. In some embodiments, the insertion occurs in a manner that is temporally aligned with the active patient context so that the guidance is presented when it is relevant to the physician's current workflow. In some embodiments, the insertion is performed in response to a clinical workflow transition, including chart open, diagnosis entry, allergy entry, organ function change (e.g. ejection fraction, pulmonary function or GFR), or medication ordering, so that the guidance is not presented in a manner that interrupts the physician's completion of higher-priority clinical tasks.

In some embodiments, the system utilizes a system for improving patient outcomes to determine what constitutes a higher-priority clinical task. In some embodiments, the guidance is inserted only when the system determines that the guidance is more clinically relevant than generic alerts or background recommendations. In some embodiments, the system determines that such guidance is more clinically relevant based on local patient-relevance filters that evaluate comparative relevance scores between generalized guidelines and patient-specific predictive insights, and the system prioritizes the higher-relevance content. In some embodiments, messaging is inserted only when predicted clinical value exceeds a predetermined threshold to reduce alert fatigue.

In some embodiments, the system computes a relevance score for candidate medical information, recommendations, or replacement media using a weighted aggregation of multiple relevance factors. Each relevance factor represents a distinct dimension of clinical, contextual, or operational significance associated with an active patient context.

In some embodiments, relevance factors include one or more patient state factors, including current diagnoses, comorbidities, laboratory values, imaging findings, medication history, risk classifications, or observed physiological trends derived from the clinical feed. In some embodiments, relevance factors further include treatment context factors, including care setting, encounter type, stage of treatment, acuity level, and temporal proximity to clinical workflow events such as order entry, diagnosis entry, acuity setting change or discharge planning.

In some embodiments, relevance factors further include provider-related factors, including provider specialty, role, historical interaction patterns with prior recommendations, and observed acceptance, modification, deferral, or override behavior. In some embodiments, relevance factors further include operational resource factors, including availability of medications, devices, staffing, or services within a local healthcare environment. In some embodiments, relevance factors further include geographic or epidemiological factors, including regional disease prevalence, outbreak conditions, formulary variation, or location-specific practice constraints.

In some embodiments, each relevance factor is associated with a weighting factor, and the relevance score is computed as a weighted combination of the relevance factors. In some embodiments, weighting factors are dynamically adjustable over time based on observed clinician interaction events and patient outcomes associated with previously delivered recommendations.

In some embodiments, adaptive adjustment of weighting factors is performed by a calibration process that evaluates correlations between relevance factors, clinician responses, and downstream patient outcomes, and adjusts one or more weighting factors accordingly. In some embodiments, safeguards are applied to adaptive adjustment, including bounding the magnitude of weight changes, enforcing minimum data thresholds before adjustment, limiting adjustment frequency, maintaining baseline configurations, and reverting adjustments when instability, degraded performance, or increased alert fatigue is detected.

In some embodiments, the computed relevance score is compared to one or more predetermined or adaptively calibrated thresholds to determine whether a candidate recommendation is selected for insertion, as well as to determine an appropriate workflow insertion modality, timing, or presentation format.

In some embodiments, the system determines whether to insert, replace, or present replacement media within a clinical workflow based on a comparison of a computed relevance score to a relevance threshold. In some embodiments, the replacement media is inserted into the workflow only when the relevance score satisfies the relevance threshold, thereby preventing presentation of low-value or contextually irrelevant information.

In some embodiments, when the relevance score does not meet the relevance threshold, the system refrains from overriding the clinical feed and leaves the clinical feed unmodified for that workflow instance. In some embodiments, when the relevance score does not meet the relevance threshold, the system suppresses insertion of the replacement media, defers presentation to a later workflow stage, or makes the replacement media available only through a non-interruptive or background interface. In this manner, the system reduces unnecessary alerts and minimizes disruption to clinical reasoning while preserving access to potentially useful information.

In some embodiments, the relevance threshold is configurable and may vary based on care setting, workflow stage, provider role, or risk classification of the active patient context reflected in the clinical feed. In some embodiments, the relevance threshold is adaptively calibrated over time based on observed clinician interaction patterns, including acceptance, modification, deferral, or override behavior, and based on downstream patient outcomes associated with previously inserted replacement media.

In some embodiments, adaptive calibration of the relevance threshold is subject to one or more safeguards, including bounding the rate or magnitude of threshold adjustment, requiring a minimum volume of interaction data prior to adjustment, maintaining baseline threshold values, and reverting threshold changes when increased alert fatigue or degraded clinical utility is detected.

In some embodiments, the relevance threshold functions as a gating mechanism that controls workflow insertion, such that only replacement media exceeding a minimum relevance criterion is eligible for insertion or replacement within the clinical workflow. This threshold-based gating improves user experience by prioritizing clinically meaningful, patient-specific content derived from the clinical feed and reducing low-value interruptions.

In some embodiments, when no candidate replacement media item satisfies the predetermined threshold, the system refrains from overriding the clinical feed and leaves the clinical feed unmodified for that workflow instance. In some embodiments, when the relevance score does not satisfy the predetermined threshold, the system suppresses presentation of the candidate replacement media, defers presentation until a later workflow transition, or makes the candidate replacement media available only through a non-interruptive interface location, thereby reducing low-value alerts and improving clinician user experience.

This allows clinical content to be preserved and respected while enabling selective augmentation with patient-relevant information that improves diagnostic accuracy, treatment personalization, and care outcomes. In some embodiments described herein, the term system for improving patient outcomes refers to a clinical decision support system that provides timely and person-specific information, intelligently filtered or presented at appropriate times, to enhance patient outcomes and quality of care.) In some embodiments, the system for improving patient outcomes operates as a component of an electronic health record system or as a stand-alone system or plug-in to an electronic health record system. In some embodiments, the system for improving patient outcomes combines medical knowledge that can be used by computers with information specific to an individual patient to give useful information in real time. In some embodiments, the system for improving patient outcomes reduces errors and adverse events and improves patient safety, including preventing medication interactions through automated checks. In some embodiments, the system for improving patient outcomes determines what constitutes a higher-priority clinical task by determining that a task is associated with improved patient safety or improved patient outcomes relative to other tasks in the physician's workflow, and the system utilizes that determination to avoid disrupting higher-priority clinical tasks when inserting contextualized, patient-specific guidance into the physician's workflow.

To increase robustness, embodiments herein deliberately prioritize clinically interpretable data and observable patient indicators rather than relying solely on metadata or administrative tags. In some embodiments, the clinically interpretable data and observable patient indicators are derived from the clinical feed, including structured and unstructured clinical data, and include information that is relevant to clinical decision making and patient safety. In some embodiments, the clinically interpretable data includes at least one of clinical encounter details, diagnoses, symptoms, treatments, laboratory test results, prescriptions, imaging, charts, patient experience insights, and clinical narratives, and the system prioritizes such data when determining, based on the clinical feed, a categorization of the clinical feed. In some embodiments, the system provides timely and person-specific information, intelligently filtered or presented at appropriate times, to enhance patient outcomes and quality of care, and the deliberate prioritization of clinically interpretable data is used to improve the reliability of categorization and the relevance of replacement media. For example, a patient may not be explicitly labeled as “high risk,” but predictive modeling based on lab trends, comorbidities, and behavioral markers may identify elevated risk within the boundaries of an active encounter.

In some embodiments, such elevated risk is identified without reliance on administrative tags by evaluating observable patient indicators present in the clinical feed, and the system uses the resulting categorization to insert contextualized, patient-specific guidance into the physician's workflow as described herein. In some embodiments, the system for improving patient outcomes utilizes the clinically interpretable data and observable patient indicators from the clinical feed to determine what constitutes a higher-priority clinical task and to ensure that insertion of contextualized guidance is intelligently filtered or presented at appropriate times without disrupting higher-priority clinical tasks.

Medical insights, candidacy for research trials are delivered to physicians may be intelligently selected based on a number of factors. For example, patient-specific clinical history; genomic or biomarker information; real-time lab values; medication interactions; social determinants of health; behavioral and adherence data; time-sensitive clinical guidelines; and physician specialty and treatment style. In some embodiments, the factors are derived from the clinical feed associated with an active patient context, and the system evaluates the factors together to determine, based on a set of information, a replacement media that includes patient-specific medical information and recommendations delivered to a physician at the point of care. In some embodiments, the medical insights correspond to clinical decision support that provides knowledge and person-specific information, intelligently filtered or presented at appropriate times, to enhance health and health care, and the replacement media includes at least one of clinical guidelines, condition-specific order sets, focused patient data reports and summaries, documentation templates, diagnostic support, or contextually relevant reference information.

Embodiments herein support dynamic insertion of patient-specific guidance into existing clinical workflows when the system determines that such guidance is more clinically relevant than generic alerts or background recommendations. In some embodiments, the system determines that the guidance should be inserted at an appropriate time and at a point in workflow so that the physician can act on the information quickly and confidently, and the system avoids presenting guidance in a manner that contributes to alert fatigue. Embodiments herein include local patient-relevance filters that evaluate comparative relevance scores between generalized guidelines and patient-specific predictive insights, prioritizing the higher-relevance content. In some embodiments, the local patient-relevance filters implement a right information and right time approach by selecting which content is inserted and when it is inserted based on the active patient context reflected in the clinical feed. In some embodiments, the system for improving patient outcomes utilizes outputs of the local patient-relevance filters to prioritize the higher-relevance content for insertion into the physician's workflow, thereby improving patient safety and reducing adverse events while avoiding disruption of higher-priority clinical tasks.

In some embodiments, the local patient-relevance filters evaluate comparative relevance scores between generalized guidelines and patient-specific predictive insights by computing a relevance score for a candidate replacement media that reflects a predicted clinical value of inserting the candidate replacement media into a physician workflow in view of an active patient context reflected in the clinical feed. In some embodiments, the relevance score is computed using a combination of computable medical knowledge and information specific to an individual patient so that the system provides knowledge and person-specific information that is intelligently filtered or presented at appropriate times to enhance health and health care. In some embodiments, the relevance score is computed as a weighted combination of factor scores derived from the clinical feed and from the set of information, where each factor score is mapped to a common range and the weights reflect relative contribution to predicted clinical value. In some embodiments, the factor scores include a patient state score reflecting an emerging risk state derived from symptoms, lab trends, imaging, medication history, behavioral indicators, and risk patterns; a guideline urgency score reflecting time-sensitive clinical guidelines applicable to the active patient context; a medication interaction score reflecting an interaction risk between a current medication regimen and a contemplated medication adjustment; a specialty match score reflecting physician specialty and treatment style; a social risk score reflecting social determinants of health and behavioral and adherence data; and an operational availability score reflecting availability of medicines based on stock levels of pharmaceuticals and locations of medicines.

In some embodiments, the relevance score is determined using operational resource data together with patient-specific clinical data so that the system prioritizes replacement media that is both clinically indicated and operationally feasible in the active patient context. In some embodiments, the operational resource data includes one or more of pharmaceutical inventory data, device availability data, and care team capacity data. In some embodiments, pharmaceutical inventory data includes stock levels, locations of medicines, formulary constraints, or replenishment status. In some embodiments, device availability data includes availability of diagnostic devices, therapeutic devices, monitoring devices, or other clinical equipment relevant to execution of a recommended action. In some embodiments, care team capacity data includes availability, workload, or scheduling constraints of physicians, nurses, pharmacists, specialists, technicians, or other care personnel involved in carrying out the recommended action. In some embodiments, the operational resource data contributes to an operational resource score or one or more operational sub-scores that are aggregated with clinical factor scores in determining the relevance score.

In some embodiments, the relevance score computation includes explicit weighting factors, and the weighting factors are selected so that patient safety and higher-severity risks contribute more strongly to relevance than lower-severity risks. In some embodiments, the weighting factors include a patient state weight of approximately 0.35, a medication interaction weight of approximately 0.20, a guideline urgency weight of approximately 0.15, a lab trend and biomarker weight of approximately 0.10, a social risk and adherence weight of approximately 0.10, a specialty match weight of approximately 0.05, and an operational availability weight of approximately 0.05, with the weights normalized so that the weighted combination produces a relevance score on a predetermined scale. In some embodiments, different weight sets are used for different clinical contexts, including inpatient, outpatient, telehealth, and home-based care, and the system selects the weight set based on the categorization of the clinical feed. These weights can be predetermined or defined dynamically by the system based upon feedback and patient outcome improvements.

In some embodiments, thresholding is applied to the relevance score such that insertion occurs only when the relevance score exceeds a predetermined threshold. In some embodiments, the predetermined threshold is selected to reduce alert fatigue and to avoid presenting guidance in a repeated and non-relevant manner that leads to deferral, ignoring, or override. In some embodiments, the predetermined threshold is selected using a right information and right time approach in which the system selects the point in workflow and the channel of presentation that supports completion of the right action without disrupting higher-priority clinical tasks. In some embodiments, the predetermined threshold is different for different intervention formats, including a threshold for interruptive insertion in an electronic health record user interface, a threshold for non-interruptive presentation within a clinical decision-support dashboard, and a threshold for presentation within a mobile clinical device interface, and the system selects among these thresholds based on the system for improving patient outcomes determining what constitutes a higher-priority clinical task for the active patient context.

In some embodiments, calibration includes adjusting the relevance score computation, the weighting factors, or the predetermined threshold based on observed clinical workflow behavior and observed outcomes. In some embodiments, the observed clinical workflow behavior includes whether a physician takes an action in response to inserted guidance, whether the physician accepts, modifies, defers, or provides feedback, and whether inserted guidance is ignored or overridden. In some embodiments, the observed outcomes include patient outcome indicators monitored by the system for improving patient outcomes, including whether predicted elevated risk is confirmed or avoided, whether medication interaction risk is reduced, and whether time-sensitive clinical guidance is followed during an active encounter. In some embodiments, calibration is performed in stages, including an initial calibration based on retrospective clinical feed data and historical outcomes data, followed by an operational calibration during deployment in which the system periodically updates calibration parameters based on newly observed workflow behavior and outcomes data. In some embodiments, the system performs calibration by adjusting one or more weighting factors to increase the relevance contribution of factors that are associated with improved patient outcomes and to decrease the relevance contribution of factors that are associated with repeated deferral or override without corresponding clinical benefit. In some embodiments, the system performs calibration by adjusting the predetermined threshold upward in response to excessive insertion frequency or excessive override, and by adjusting the predetermined threshold downward in response to missed opportunities to provide clinically relevant guidance for higher-severity risk states. In some embodiments, adaptive calibration is constrained by one or more safeguards, including limiting an amount of change to the weighting factors or the predetermined threshold within a predetermined period, limiting calibration to occur only after a minimum amount of observed workflow behavior is available, and maintaining a record of calibration changes and associated observed outcomes so that changes can be reviewed and reversed if needed.

In some embodiments, the weighting factors that are eligible to be adaptive include a patient state weight corresponding to an emerging risk state derived from symptoms, lab trends, imaging, medication history, behavioral indicators, and risk patterns; a medication interaction weight corresponding to interaction risk between current medications and contemplated medication adjustments; a guideline urgency weight corresponding to time-sensitive clinical guidelines applicable to the active patient context; a lab trend and biomarker weight corresponding to changes in real-time lab values, including deviations from a baseline or from an expected trajectory; a social risk and adherence weight corresponding to social determinants of health and behavioral and adherence data; a specialty match weight corresponding to physician specialty and treatment style; and an operational availability weight corresponding to availability of medicines based on stock levels of pharmaceuticals and locations of medicines. In some embodiments, each eligible weighting factor is bounded by a minimum value and a maximum value, and the eligible weighting factors are normalized so that a weighted combination of factor scores produces a relevance score on a predetermined scale.

In some embodiments, adaptive changes to the weighting factors are triggered by clinical feed conditions that indicate a change in the active patient context. In some embodiments, the triggers include a categorization of the clinical feed indicating an emerging risk state, an early sepsis risk, a medication interaction risk, or a deteriorating heart failure indicator; a change in real-time lab values that crosses a predetermined boundary or exhibits a predetermined trend pattern; an update to medication history indicating initiation, discontinuation, or dose adjustment of a medication associated with interaction risk; receipt of imaging results or imaging reports that indicate a change in patient state; receipt of behavioral indicators or adherence data indicating increased risk; receipt of updated social determinants of health indicating a change in patient context; receipt of updated time-sensitive clinical guidelines indicating a change in applicable guidance; and receipt of operational medical information indicating that stock levels of pharmaceuticals or locations of medicines constrain an available treatment option. In some embodiments, the triggers include clinical workflow transitions reflected in the clinical feed, including chart open, diagnosis entry, medication ordering, and/order signing, and the system adjusts weighting factors so that the right information is presented at the right time in the workflow. In some embodiments, the operational medical information further includes device availability information and care team capacity information associated with a hospital system, outpatient clinic, telehealth environment, or other care setting relevant to the active patient context.

In some embodiments, the weighting factors may be continuously adaptive. In some embodiments, the system updates one or more eligible weighting factors on a rolling basis using a sliding window of recent clinical feed data and recent physician interaction data so that the weighting factors reflect current clinical context and current workflow behavior. In some embodiments, the system updates the weighting factors periodically at a predetermined interval. In some embodiments, the system updates the weighting factors in response to an event-driven trigger in the clinical feed. In some embodiments, the system selects among continuous adaptive updating, periodic updating, or event-driven updating based on a care setting, a categorization of the clinical feed, or a severity of the active patient context.

In some embodiments, safeguards are applied to adaptive changes to the weighting factors. In some embodiments, the safeguards include limiting an amount of change to any eligible weighting factor within a predetermined period, limiting an amount of change per update, and limiting adaptive changes to occur only after a minimum amount of observed clinical workflow behavior is available. In some embodiments, the safeguards include maintaining a record of adaptive changes, including a record of prior weighting factor values, updated weighting factor values, and clinical feed conditions that triggered the adaptive changes, so that adaptive changes can be reviewed and reversed if needed. In some embodiments, the safeguards include maintaining a baseline set of weighting factors and reverting to the baseline set of weighting factors when a trigger condition indicates degraded performance, including increased alert fatigue, increased override, or decreased action in response to inserted guidance. In some embodiments, the safeguards include separating weighting factors for different intervention formats so that a weighting factor adjustment that would increase interruptive insertion does not automatically increase interruptive insertion unless the system also determines that the insertion will not disrupt higher-priority clinical tasks. In some embodiments, the safeguards include requiring that adaptive changes remain within predetermined bounds that preserve prioritization of patient safety and higher-severity risk states relative to lower-severity risk states. In some embodiments, the safeguards include maintaining normalization constraints so that the weighting factors continue to sum to a predetermined total or remain within a predetermined range. In some embodiments, the system for improving patient outcomes utilizes the clinical feed conditions and the observed workflow behavior to determine whether adaptive changes to weighting factors should be applied, and utilizes the safeguards to reduce alert fatigue while ensuring that contextualized, patient-specific guidance remains intelligently filtered or presented at appropriate times.

In some embodiments, embodiments herein include transmission of individualized clinical recommendations via secure digital channels for integration into EHR interfaces, mobile clinical devices, or decision-support dashboards without interrupting primary care delivery. In some embodiments, the secure digital channels include secure transmission of information derived from the clinical feed, including patient-specific clinical information, more general medical information, or operational medical information, and the secure digital channels are configured to protect confidentiality and integrity of the transmitted information.

In some embodiments, secure channel properties include encryption of data in transit, integrity protection, and replay protection, such that the transmitted clinical recommendations and associated context are resistant to interception, alteration, or reuse. In some embodiments, secure channel properties include a negotiated session with ephemeral session keys and message authentication so that the secure channel provides confidentiality and integrity for each transmitted message. In some embodiments, secure channel properties include binding a message or a session to an active patient context so that a clinical recommendation is associated with a particular patient context and is not misapplied outside that context.

In some embodiments, authentication includes verifying an identity of a clinician interface device, an EHR interface, a decision-support dashboard, a remote clinical intelligence server, or another endpoint participating in transmission of clinical recommendations. In some embodiments, authentication includes certificate-based authentication, token-based authentication, or a combination thereof, and the system establishes a communication session only when authentication is successful. In some embodiments, authentication includes verifying that an authenticated endpoint is associated with a permitted organization, permitted department, permitted care setting, or permitted workflow role.

In some embodiments, access control includes role-based access control or attribute-based access control, or both, such that access to clinical recommendations, the clinical feed, or both, is limited to authorized clinicians, authorized staff, or authorized systems. In some embodiments, access control includes least-privilege constraints so that an authenticated endpoint receives only a minimum necessary subset of information required to present the clinical recommendation in the physician workflow. In some embodiments, access control includes contextual access checks based on at least one of a clinician role, a physician specialty, an encounter type, a care setting, or a workflow state reflected in the clinical feed. In some embodiments, access control includes policy rules that restrict access to specific types of clinical feed information, including restricting access to patient identifiers, restricting access to portions of unstructured clinical documentation, or restricting access to operational medical information such as stock levels of pharmaceuticals and locations of medicines to authorized operational roles.

In some embodiments, audit logging includes generating and maintaining an audit record for access to the clinical feed, selection of replacement media, insertion of contextualized guidance, clinician interaction events, and changes to relevance score parameters, thresholds, weighting factors, or adaptive calibration settings. In some embodiments, the audit record includes an identifier of a user, device, or system component performing an action, a timestamp, an active patient context identifier or pseudonymized equivalent, a description of the action performed, and one or more identifiers of affected recommendations, rules, templates, or model versions. In some embodiments, the audit records are stored in a tamper-evident or append-only log and are used to support traceability, rollback, compliance review, patient-safety review, and reproducibility of workflow insertion decisions.

In some embodiments, privacy controls include minimizing transmission of patient-identifying information by transmitting derived features, de-identified information, pseudonymized identifiers, or a minimum necessary subset of patient context data. In some embodiments, privacy controls include executing portions of classification, relevance scoring, or insertion logic locally on a client or on-premise computing device so that patient-specific information derived from the clinical feed is not transmitted outside a controlled boundary. In some embodiments, privacy controls include controlling caching of clinical recommendations on a physician device, including limiting a cache duration, limiting a cache size, or invalidating cached recommendations when an active patient context ends. In some embodiments, privacy controls include applying policy constraints to prevent storage of certain clinical feed elements in logs or caches, including preventing storage of free-text clinical documentation or preventing storage of patient identifiers. In some embodiments, the secure channel properties, authentication, access control, audit logging, and privacy controls are selected so that clinical decision support provides knowledge and person-specific information that is intelligently filtered or presented at appropriate times while protecting patient privacy and supporting patient safety and improved patient outcomes.

Embodiments herein include systems compatible with existing hospital systems, outpatient clinics, telemedicine platforms, decentralized care networks, and centralized health information exchanges.

In some embodiments, the framework includes a physician device, a remote clinical intelligence server, an electronic health record (EHR) system, and secure communication interfaces. The physician device may display patient charts while simultaneously receiving patient-specific clinical insights from the remote system. In some embodiments, the method includes a number of enumerated steps, but aspects of the method may include additional steps before, after, and in between the enumerated steps. In some aspects, one or more of the enumerated steps may be omitted or performed in a different order.

At step 701, a system receives a clinical feed. The clinical feed includes clinical information associated with an active patient context, including structured and unstructured clinical data. At step 702, the system determines based on the clinical feed, a categorization of the clinical feed. In some embodiments, the categorization indicates a patient state or a clinical context within an active encounter. The system may integrate with the EHR to monitor active patient encounters. A clinical classifier may analyze incoming patient data streams, including labs, vitals, imaging reports, and notes, to determine emerging clinical states, including early sepsis risk, medication interaction risk, and deteriorating heart failure indicators.

At step 703, the system determines, based on a set of information, a replacement media. In a medical embodiment, the replacement media includes patient-specific medical information and recommendations delivered to the physician. In some embodiments, the system for improving patient outcomes utilizes the set of information and the clinical feed to determine which replacement media is expected to enhance patient outcomes and quality of care when inserted into the physician's workflow.

In some embodiments, determining the replacement media includes generating or identifying a candidate set of replacement media items that are eligible for insertion into the physician's workflow. In some embodiments, the candidate set includes at least one of suggested diagnostic tests, personalized medication adjustments, evidence-based treatment options tailored to the patient's characteristics, risk stratification insights, preventive care reminders, clinical guidelines, condition-specific order sets, focused patient data reports and summaries, documentation templates, diagnostic support, and contextually relevant reference information that are tailored to the active patient context reflected in the clinical feed. In some embodiments, the candidate set also includes content derived from more general medical information, including treatment strategies and medical journals, and the candidate set includes guidance that reflects operational medical information associated with availability of medicines, including stock levels of pharmaceuticals and locations of medicines, availability of medical hardware, equipment, when such operational medical information affects an available treatment option. In some embodiments, when operational resource data indicates that a recommended action is not currently feasible, the system decreases the relevance score for replacement media associated with that action, selects an alternative replacement media associated with a feasible action, or changes a workflow insertion modality or intervention format so that the physician receives clinically useful information without a low-value interruption.

In some embodiments, the system determines the replacement media by computing, for each candidate replacement media item, a relevance score that reflects a predicted clinical value of inserting that candidate item into the physician's workflow in view of the active patient context reflected in the clinical feed. In some embodiments, the relevance score computation is configured so that the system provides knowledge and person-specific information that is intelligently filtered or presented at appropriate times to enhance patient outcomes and quality of care. In some embodiments, the relevance score is computed as a weighted combination of factor scores derived from the clinical feed and the set of information, and the weighted combination is normalized to a predetermined scale. In some embodiments, the factor scores include a patient state score, a medication interaction score, a guideline urgency score, a lab trend and biomarker score, a social risk and adherence score, a specialty match score, and an operational availability score, and the weighting factors applied to such factor scores are selected so that patient safety and higher-severity risks contribute more strongly to relevance than lower-severity risks.

In some embodiments, the system applies thresholding to the relevance score such that a candidate replacement media item is selected for insertion only when its relevance score exceeds a predetermined threshold, and the predetermined threshold is selected to reduce alert fatigue and to avoid repeated and non-relevant insertion that leads to deferral, ignoring, or override. In some embodiments, when multiple candidate replacement media items exceed the predetermined threshold, the system ranks the candidate replacement media items based on the relevance scores and selects a highest-ranked candidate replacement media item as the replacement media. In some embodiments, the system selects among multiple candidates by selecting a candidate whose intervention format and point in workflow are determined to be suitable for insertion without disrupting higher-priority clinical tasks, as determined by the system for improving patient outcomes.

In some embodiments, the system determines the replacement media by first determining, based on the clinical feed, a categorization of the clinical feed that indicates a patient state or clinical context within an active encounter, and then using the categorization to select which candidate replacement media items to consider or to adjust weighting factors applied to factor scores for relevance scoring. In some embodiments, when the categorization indicates an emerging risk state or other higher-severity risk state, the system increases a weighting factor for a patient state score or a medication interaction score and decreases a weighting factor for lower-priority factors so that the replacement media is selected to address the higher-severity risk state. In some embodiments, when the categorization indicates that operational availability constrains treatment options, the system increases a weighting factor for an operational availability score so that the replacement media reflects availability of medicines in view of stock levels of pharmaceuticals and locations of medicines.

In some embodiments, the replacement media includes suggested diagnostic tests, personalized medication adjustments, evidence-based treatment options tailored to the patient's characteristics, risk stratification insights, and preventive care reminders specific to that patient. In some embodiments, the replacement media includes a physician-readable summary that incorporates patient-specific values from the clinical feed. In some embodiments, rather than generating an entire physician-readable summary, only a portion of the physician-readable summary is generated on the fly, including inserting specific patient lab values, risk percentages, or medication names into a pre-validated clinical explanation. In some embodiments, the replacement media includes, or is determined based on, treatment strategies or medical journals, and the system uses such more general medical information to select or contextualize patient-specific guidance delivered to the physician. In some embodiments, the replacement media includes, or is determined based on, stock levels of pharmaceuticals and locations of medicines, and the replacement media provides guidance that reflects availability of medicines in view of the active patient context.

In some embodiments, the set of information includes patient-specific clinical history; genomic or biomarker information; real-time lab values; medication interactions; social determinants of health; behavioral and adherence data; time-sensitive clinical guidelines; and physician specialty and treatment style. In some embodiments, the set of information is derived from the clinical feed associated with the active patient context, and the system evaluates the set of information together with the categorization of the clinical feed to determine a replacement media that is more clinically relevant than generic alerts or background recommendations. In some embodiments, the set of information includes treatment strategies and medical journals and includes operational medical information including stock levels of pharmaceuticals and locations of medicines, and the system uses such information in relevance score computation and ranking for determining which replacement media to deliver. In some embodiments, the set of information is retrieved from a central data server.

At step 704, the system overrides the clinical feed with the replacement media based on the categorization. In a medical embodiment, overriding includes inserting contextualized guidance into the physician's workflow when predicted clinical value exceeds a predetermined threshold to reduce alert fatigue. In some embodiments, clinical decision support provides knowledge and person-specific information that is intelligently filtered or presented at appropriate times, and overriding the clinical feed includes presenting the replacement media at the appropriate time and at a point in workflow so that the physician can act on the information quickly and confidently. In some embodiments, alert fatigue occurs when a clinician, after receiving too many alerts or reminders, begins to override or ignore further alerts without attending to them, and the predetermined threshold and insertion modality are selected to reduce such overriding or ignoring.

In some embodiments, when predicted clinical value does not exceed the predetermined threshold, overriding does not occur and the system maintains presentation of the underlying clinical feed without insertion of replacement media, thereby avoiding unnecessary interruptions and limiting alert burden.

In some embodiments, overriding based on the categorization includes selecting a workflow insertion modality that corresponds to a right channel and a right intervention format for the active patient context. In some embodiments, the workflow insertion modality includes insertion within an electronic health record user interface. In some embodiments, the workflow insertion modality includes insertion within a clinical decision-support dashboard. In some embodiments, the workflow insertion modality includes insertion within a mobile clinical device interface. In some embodiments, the system selects among these modalities based on the categorization of the clinical feed, the predicted clinical value reflected in the relevance score, and a determination by the system for improving patient outcomes of what constitutes a higher-priority clinical task in the physician's workflow.

In some embodiments, insertion within an electronic health record user interface includes presenting the replacement media as an interruptive activity, an information display or link, or targeted highlighting of relevant data. In some embodiments, the insertion is non-interruptive and remains available within the electronic health record user interface so that the physician can defer interaction with the inserted guidance and return to it at a time that is appropriate in the physician's workflow. In some embodiments, insertion within an electronic health record user interface includes presenting guidance when a patient chart is opened or when a workflow transition occurs so that the guidance is provided at a right point in workflow.

In some embodiments, insertion within a clinical decision-support dashboard includes presenting focused patient data reports, summaries, or dashboards that surface the replacement media as contextualized guidance associated with the active patient context reflected in the clinical feed. In some embodiments, the dashboard insertion is used when the predicted clinical value exceeds a dashboard threshold that is lower than a threshold used for interruptive presentation, so that the system provides clinically relevant information while reducing unnecessary interruptions. In some embodiments, insertion within a mobile clinical device interface includes providing the replacement media through a mobile channel as a right channel for clinical decision support, including when the physician is away from a workstation and the mobile clinical device interface is used as a workflow endpoint for receiving contextualized guidance. In some embodiments, the mobile insertion modality is selected when the categorization indicates that time-sensitive clinical guidance should be presented and a mobile channel is a suitable channel for presenting such guidance at an appropriate time.

In some embodiments, overriding includes applying a lockout period or other frequency control to reduce repeated presentation of the same or similar guidance in a manner that contributes to alert fatigue. In some embodiments, a lockout time is increased to reduce the number of interruptive presentations, while maintaining a non-intrusive location for guidance so that a clinician can return to the guidance at a time that is appropriate in their workflow. In some embodiments, the lockout period, the predetermined threshold, or both, are adjusted by calibration as described herein in response to observed deferral, ignoring, or override behavior so that the system continues to present the right information at the right time while reducing alert fatigue. In some embodiments, the workflow insertion modality is selected such that insertion occurs within an electronic health record user interface, within a clinical decision-support dashboard, or within a mobile clinical device interface, and the system selects an intervention format and a point in workflow consistent with providing the right information through the right channels in the right intervention formats at the right points in workflow.

Patient-specific recommendations may be generated from a remote clinical knowledge database containing evidence-based guidelines, clinical trial data, real-world outcomes data, predictive models, and text-to-speech or natural language generation modules for generating physician-readable summaries. In some embodiments, the remote clinical knowledge database stores medical knowledge usable by computers together with reference information that supports clinical decision making, and the remote clinical knowledge database is accessed to provide knowledge and person-specific information that is intelligently filtered or presented at appropriate times to enhance health and health care. In some embodiments, the remote clinical knowledge database includes one or more repositories of clinical guidelines, condition-specific order sets, focused patient data reports and summaries, documentation templates, diagnostic support, and contextually relevant reference information, and the system selects among such content to generate the patient-specific recommendations delivered to a physician at the point of care. In some embodiments, the remote clinical knowledge database includes more general medical information, including treatment strategies and medical journals, and the system uses such more general medical information to support selection of patient-specific recommendations that are relevant to an active patient context reflected in the clinical feed. In some embodiments, the remote clinical knowledge database includes, or is associated with, operational medical information including stock levels of pharmaceuticals and locations of medicines, and the system uses such information to determine whether a recommended treatment option is available at a relevant care location. In some embodiments, operational medical information included in, or associated with, the remote clinical knowledge database is obtained from one or more inventory management systems, including automated dispensing cabinet systems that monitor inventory and provide real-time stock level visibility and expiration tracking, and pharmacy inventory management systems that maintain real-time or near real-time stock positions based on dispensing, returns, and replenishment events. In some embodiments, locations of medicines are obtained from a materials management system or medication storage management system that maintains physical location attributes for items and synchronizes those location attributes with clinical systems, and stock levels are updated in response to inventory depletion events, dispensing events, restocking events, transfers between locations, and periodic reconciliation updates. In some embodiments, the remote clinical knowledge database updates the operational medical information continuously, periodically at a predetermined interval, or in response to event-driven triggers, and the system selects among continuous, periodic, or event-driven updating based on a care setting, a categorization of the clinical feed, or a time-sensitive medication availability condition reflected in the clinical feed.

In some embodiments, generating patient-specific recommendations includes determining, based on the clinical feed and a set of information, a candidate set of recommendation content items from the remote clinical knowledge database and then selecting one or more of the candidate content items as a replacement media for insertion into the physician workflow. In some embodiments, selection includes computing a relevance score for each candidate content item, applying thresholding, and selecting a highest-ranked candidate content item as described herein. In some embodiments, the physician-readable summaries are generated to include patient-specific context derived from the clinical feed so that the summary is tailored to the active patient context and is presented in a manner that supports completion of the right action at a right point in workflow.

In some embodiments, rather than generating an entire recommendation, only a portion of the recommendation is generated in real-time, for example inserting specific patient lab values, risk percentages, or medication names into a pre-validated clinical explanation. In some embodiments, the pre-validated clinical explanation includes fixed explanatory content that is not altered by the real-time generation, and the real-time generation is limited to insertion of patient-specific values or terms derived from the clinical feed into predetermined insertion locations within the pre-validated clinical explanation. In some embodiments, the real-time generation includes generating a physician-readable summary portion that is formatted for presentation within an electronic health record user interface, within a clinical decision-support dashboard, or within a mobile clinical device interface, and the system selects the presentation channel and intervention format based on predicted clinical value and a predetermined threshold to reduce alert fatigue.

In some embodiments, the real-time generation includes selecting which patient-specific values to insert based on the categorization of the clinical feed and based on local patient-relevance filters, such that the inserted values correspond to the factors that contributed to the relevance score for selecting the recommendation. In some embodiments, the real-time generation includes inserting an indication of medication interaction risk, an indication of an emerging risk state, or an indication of time-sensitive clinical guidance applicability, where such indications are derived from the clinical feed and are inserted into the pre-validated clinical explanation without altering the fixed explanatory content.

In some embodiments, physicians, nurses, and other medical personnel provide patient notes to the system by dictating narrative clinical documentation, including during or immediately after an encounter, and the dictated content is received as part of the clinical feed associated with an active patient context. In some embodiments, the system performs speech-to-text transcription on the dictated content to generate editable text for inclusion in patient notes, and the system performs cleaning of the dictated content including formatting, normalization of clinical terminology, and correction of transcription artifacts so that the resulting text is suitable for entry into an electronic health record. In some embodiments, the system provides suggestions for patient notes based on information in the clinical feed, including suggesting missing elements, suggested phrasing, or clarifying language, and the system provides prewritten notes or prewritten portions of notes that are tailored to the active patient context reflected in the clinical feed. In some embodiments, the system generates preliminary clinical documentation from clinician-patient conversations using speech recognition and natural language generation, and the generated documentation is presented to the medical professional as content suggestions that can be accepted, rejected, or edited before the notes are finalized and entered into the system. In some embodiments, every generated summary statement or suggested note portion is associated with traceable transcript references so that the medical professional can verify accuracy and locate a source of the suggestion prior to entering the notes into the electronic health record.

In some embodiments, the pre-validated clinical explanation is curated, validated, and updated to preserve clinical content integrity while enabling selective augmentation with patient-specific information. In some embodiments, curation includes creating and maintaining a library of pre-validated clinical explanations stored in the remote clinical knowledge database, where each pre-validated clinical explanation is associated with an identifier and a version. In some embodiments, curation includes associating each pre-validated clinical explanation with a clinical domain or condition context, and associating each pre-validated clinical explanation with one or more applicable evidence-based guidelines, clinical trial data, or real-world outcomes data. In some embodiments, validation includes review of the explanatory content to ensure consistency with the associated evidence-based guidelines or other reference sources stored in the remote clinical knowledge database, and validation further includes confirming that the predetermined insertion locations are configured so that insertion of patient-specific values does not alter the meaning of the fixed explanatory content,

In some embodiments, updating includes updating a pre-validated clinical explanation when an associated guideline changes, when clinical trial data changes, when real-world outcomes data changes, or when the system determines that a revised explanation improves clarity or clinical relevance while maintaining clinical integrity. In some embodiments, updating includes maintaining an update record that identifies a prior version and an updated version of a pre-validated clinical explanation, and maintaining a record of an effective date or applicability condition for the updated version. In some embodiments, the system selects which version of a pre-validated clinical explanation to use based on the active patient context reflected in the clinical feed and based on the time-sensitive clinical guidelines applicable to that active patient context.

In some embodiments, safeguards are applied to curation, validation, and updating of pre-validated clinical explanations. In some embodiments, safeguards include restricting who can modify a pre-validated clinical explanation, maintaining access control for modification operations, and maintaining audit logging for changes. In some embodiments, safeguards include retaining prior versions to support rollback and to support review of differences between versions. In some embodiments, safeguards include separating fixed explanatory content from inserted patient-specific values so that only predetermined insertion locations are populated by the real-time generation.

The system may also incorporate geographic epidemiological data, local outbreak trends, environmental risk factors, and regional healthcare utilization patterns to further tailor physician messaging to the patient context. In some embodiments, geographic epidemiological data and local outbreak trends include notifiable disease surveillance data that is collated and published on a recurring basis, including weekly case tables for selected infectious national notifiable diseases, and the system uses such data to adjust relevance scoring, prioritization, or recommended actions for an active patient context reflected in the clinical feed. In some embodiments, geographic epidemiological data and local outbreak trends also include outbreak notifications and outbreak lists published by public health authorities, and the system uses such outbreak information to tailor physician messaging to a patient's location or travel related context. In some embodiments, environmental risk factors include real-time or forecast environmental data, including air quality observations and forecasts, and the system uses such environmental risk factors to tailor physician messaging for patients with conditions that are sensitive to environmental conditions. In some embodiments, regional healthcare utilization patterns include hospital utilization measures, including facility-level utilization aggregated on a weekly basis, and the system uses such utilization patterns to tailor physician messaging and operational recommendations to the patient context, including when availability of care resources affects recommended actions. In some embodiments, the system integrates one or more of these data types as part of the set of information used to determine replacement media, and the system for improving patient outcomes uses the resulting context to prioritize which patient-specific guidance is inserted into the physician's workflow and when it is inserted.

In some embodiments, the data sources for geographic epidemiological data and local outbreak trends include public health surveillance datasets that are published weekly and subject to ongoing revision and delayed reporting, and the system retrieves updated values when newly published weekly tables become available. In some embodiments, the data sources for outbreak trends include outbreak notices and outbreak lists published by public health authorities, including global outbreak notices and regional epidemiological alerts, and the system retrieves updated outbreak information when new notices are published or when an existing notice is updated. In some embodiments, the data sources for environmental risk factors include air quality feeds that receive real-time observations and provide forecasts, and the system retrieves updated air quality information on an hourly basis or another periodic basis consistent with the feed. In some embodiments, the data sources for environmental risk factors include weather alerts, observations, or forecasts provided via a weather data service, and the system retrieves updated weather information based on a cache-friendly expiration schedule and forecast office updates, including updates that occur at least once every six hours in some implementations. In some embodiments, the data sources for regional healthcare utilization patterns include facility-level hospital utilization data aggregated on a weekly basis, and the system retrieves updated utilization patterns on a weekly update cadence but any temporal sequence can be selected.

The physician device may cache prioritized insights for rapid insertion during clinical workflow transitions, including chart open, diagnosis entry, and medication ordering. In some embodiments, the physician device includes a local cache and/or cache control that stores replacement media, physician-readable summaries, or other contextualized guidance that has been selected for an active patient context, so that the guidance is ready for insertion when a workflow transition occurs rather than requiring the system to retrieve or generate the guidance at the moment of insertion. In some embodiments, the cached content includes one or more of a selected recommendation, a precomputed relevance score, a set of factor scores used in relevance scoring, or a preformatted presentation payload suitable for insertion within an electronic health record user interface, within a clinical decision-support dashboard, or within a mobile clinical device interface. In some embodiments, the caching is performed by transmitting the selected content to the physician device before it is to be presented so that the content is cached and ready to present when triggered by a workflow transition, and the physician device uses such cached content to reduce delay and improve responsiveness during chart open, diagnosis entry, medication ordering, or order signing. In some embodiments, invalidation behavior is applied so that cached insights are not stale, including invalidating cached content when an active encounter ends, when the clinical feed changes in a manner that alters the categorization or the relevance score for the cached content, when a medication order is changed such that medication interaction risk changes, when updated lab values or imaging reports change a patient state assessment, or when time-sensitive guideline applicability changes for the active patient context. In some embodiments, invalidation behavior includes time-based expiration, encounter-based expiration, or event-driven invalidation based on updates to the clinical feed, and the system maintains a record of when content was cached and an applicability condition for using the cached content so that insertion uses only currently applicable content.

The system may learn from physician interaction patterns, treatment outcomes, and patient responses to refine future patient-specific recommendations. Behavioral modeling may incorporate physician decision preferences, patient adherence patterns, historical outcome data, and follow-up compliance. In some embodiments, the learning is implemented as a learning loop in which health information technology data is reused to measure results and to support continuous improvement, and the learning loop is configured to make the right information available to the right people at the right time while improving subsequent performance of the system.

In some embodiments, the learning loop includes logging interaction data associated with presentation and insertion of contextualized guidance, and the logged interaction data includes a recommendation identifier for the replacement media, an active patient context identifier, a timestamp of presentation, and a workflow context indicating a channel and an intervention format by which the guidance is presented. In some embodiments, the logged interaction data further includes a clinician identifier or role identifier, a care setting identifier, and an event type indicating whether the guidance is accepted, modified, deferred, provided feedback, ignored, or overridden. In some embodiments, the logged interaction data further includes a time-to-action value indicating a time between presentation and an action by the clinician, and a repetition indicator indicating whether the same or similar guidance was recently presented within a lockout period.

In some embodiments, the learning loop includes logging edit data when a clinician modifies inserted guidance or suggested documentation, and the logged edit data includes a record of edits to a physician-readable summary or a suggested note portion, including an indication of which portion of the content was edited and an indication of whether the edit corresponds to formatting cleanup, terminology normalization, omission of content, or addition of content. In some embodiments, the logged edit data is associated with the recommendation identifier and the active patient context identifier so that the system can determine which content types, formats, and channels tend to require fewer edits for a given physician, specialty, or care setting.

In some embodiments, the learning loop includes logging outcome indicators derived from subsequent updates to the clinical feed. In some embodiments, the outcome indicators include an indication that a recommended follow-up action is completed within a predetermined period, an indication of follow-up compliance, and an indication of patient adherence patterns. In some embodiments, the outcome indicators include changes in laboratory results, changes in vital signs, changes in medication orders, or other subsequent clinical feed updates that are relevant to the recommendation delivered to the physician. In some embodiments, the learning loop uses the outcome indicators to evaluate whether a delivered recommendation is associated with improved results, reduced adverse events, or reduced medication interaction risk, and the learning loop uses that evaluation to refine subsequent prioritization of replacement media.

In some embodiments, linking logged interaction data to a patient context and a recommendation includes associating each logged event with the active patient context identifier and the recommendation identifier, and further associating the logged event with an encounter identifier or workflow instance identifier. In some embodiments, the active patient context identifier is a pseudonymized identifier, and patient-identifying information is excluded from logged interaction data so that the learning loop can operate using a minimum necessary subset of information. In some embodiments, the system stores a mapping between a pseudonymized identifier and a patient identifier only within a controlled boundary and only for as long as needed to compute outcome indicators from the clinical feed.

In some embodiments, the learning loop refines future recommendations by updating at least one of relevance score computation, weighting factors, adaptive weighting factors, or predetermined thresholds used for selecting replacement media and determining when to insert contextualized guidance. In some embodiments, the learning loop increases a weighting factor for content types and factor scores that are associated with accepted recommendations and improved outcome indicators, and decreases a weighting factor for content types and factor scores that are associated with repeated deferral, ignoring, or override without corresponding improvement in outcome indicators. In some embodiments, the learning loop adjusts a predetermined threshold upward when logged interaction data indicates repeated overriding or ignoring consistent with alert fatigue, and adjusts the predetermined threshold downward when logged interaction data indicates missed opportunities to present clinically relevant guidance for higher-severity risk states.

In some embodiments, safeguards are applied to the learning loop and to updates produced by the learning loop. In some embodiments, safeguards include applying access control to restrict access to logged interaction data, applying audit logging for access to logged interaction data, and applying privacy controls that minimize storage of patient-identifying information in the logged interaction data. In some embodiments, safeguards include limiting how quickly thresholds or weighting factors may change by bounding an amount of change within a predetermined period, requiring a minimum amount of logged interaction data before applying an update, and maintaining a baseline configuration to which the system can revert. In some embodiments, safeguards include maintaining a record of updates made by the learning loop, including prior parameter values and updated parameter values, and maintaining an effective date or applicability condition for each update so that updates can be reviewed and reversed if needed. In some embodiments, safeguards include preventing the learning loop from increasing alert burden in a manner that contributes to alert fatigue, including enforcing lockout periods and frequency controls for repeat presentations.

In some embodiments, the system supports two-way interaction. For example, the system may present a recommendation and allow the physician to accept, modify, defer, or provide feedback. In some embodiments, the two-way interaction is implemented through a user interface presented within an electronic health record user interface, within a clinical decision-support dashboard, or within a mobile clinical device interface, and the system captures the physician response as structured interaction data associated with a recommendation identifier and an active patient context identifier. In some embodiments, accepting includes taking an action that is consistent with the presented recommendation, including selecting an order set, initiating an order, or inserting a suggested documentation portion into a patient note, and the system records acceptance as an interaction event for use in the learning loop described herein. In some embodiments, modifying includes editing the recommendation content, editing a physician-readable summary, editing a suggested patient note portion, or adjusting a suggested diagnostic test or medication adjustment, and the system records the modification and associated edits so that subsequent recommendations can be refined for a physician, a specialty, or a care setting. In some embodiments, deferring includes delaying action on the recommendation while maintaining the recommendation in a non-intrusive location for later review, and the system records a deferral event, including a time and workflow context, and uses the deferral event to calibrate thresholding and channel selection to reduce alert fatigue. In some embodiments, providing feedback includes entering an explicit feedback input and/or selecting an override reason, and the system stores the feedback input with the recommendation identifier and active patient context identifier so that the feedback can be used to refine relevance scoring, weighting, intervention format, or presentation timing for future recommendations. In some embodiments, the system provides traceable references for generated summaries or suggested note portions, such that each generated statement or suggested content portion is associated with a transcript reference or other source reference, and the physician can review, edit, accept, reject, or defer the suggestion prior to entry into the electronic health record.

In some embodiments, the two-way interaction described herein is an input to the learning loop and to adaptive calibration of the system. In some embodiments, each accept, modify, defer, and feedback event is captured as structured interaction data associated with a recommendation identifier and an active patient context identifier, and the structured interaction data is stored in audit logs or another log store to enable monitoring of whether clinicians act on decision support or bypass or postpone decision support. In some embodiments, the learning loop uses the structured interaction data as labeled signals to update at least one of relevance score computation, predetermined thresholding, workflow insertion modality selection, lockout timing, weighting factors, or adaptive weighting factors, so that knowledge and person-specific information is intelligently filtered or presented at appropriate times. In some embodiments, the learning loop increases a predicted clinical value or a weighting factor for content types and factor scores that are associated with acceptance and timely action, and decreases a predicted clinical value or a weighting factor for content types and factor scores that are associated with repeated deferral, ignoring, or override without corresponding clinical benefit, thereby reducing alert fatigue.

In some embodiments, modify events are used to refine future recommendations and future generated content. In some embodiments, when a physician modifies a recommendation, edits a physician-readable summary, or edits a suggested patient note portion, the system stores edit metadata identifying which portion of the content was edited and how it was edited, and the learning loop uses aggregated edit patterns to adjust future presentation format, phrasing, or selection of replacement media for a physician, a specialty, or a care setting. In some embodiments, the learning loop uses modify events to refine prewritten documentation suggestions, including selecting which pre-validated explanation template to use, selecting which predetermined insertion locations to populate, and selecting which patient-specific values to insert from the clinical feed, while maintaining that a clinician can review, edit, and finalize the content prior to entry into the electronic health record.

In some embodiments, feedback events include explicit feedback inputs or override reasons and the system stores the feedback events with the recommendation identifier and the active patient context identifier, and the learning loop uses the feedback events to refine relevance scoring, thresholding, channel selection, and intervention format selection. In some embodiments, when the system presents generated summaries or suggested note portions, each generated statement is associated with traceable transcript references or other source references so that the clinician can verify accuracy and locate the source prior to accepting, rejecting, editing, or deferring the suggestion, and the learning loop uses the resulting accept, reject, edit, and defer events to calibrate subsequent suggestions. In some embodiments, safeguards are applied to learning-loop updates driven by two-way interaction, including limiting an amount of change to thresholds or weighting factors within a predetermined period, requiring a minimum amount of interaction data before applying an update, maintaining a baseline configuration, and reverting to the baseline configuration when interaction data indicates increased alert fatigue or reduced action on decision support.

This feedback loop may be used to continuously refine patient-specific modeling. In some embodiments, the system monitors and records clinician interaction signals associated with delivered insights, recommendations, alerts, or workflow insertions (collectively, “insight items”) and uses those signals to update one or more patient-specific models, clinician-specific preference models, and/or population-level calibration parameters. For example, the system may treat clinician interactions—including acceptance, deferral, dismissal, editing, confirmation, ordering behavior, documentation behavior, and navigation behavior—as supervised or semi-supervised labels indicative of clinical relevance, urgency, and usability in the current context. The feedback loop may operate at multiple timescales, including (i) near-real-time session refinement for the current encounter, (ii) longitudinal refinement across encounters for the same patient, and (iii) periodic recalibration across multiple patients, clinicians, sites, or care settings to reduce drift and improve generalization.

In some embodiments, physician feedback is captured explicitly and/or implicitly. Explicit feedback may include, by way of example, clinician selection of an “accept,” “reject,” “snooze,” “not applicable,” or “explain why” control; free-text comments; structured reason codes (e.g., contraindication present, already addressed, outside scope, false positive, insufficient evidence); or edits to a proposed order set, assessment, plan, or documentation snippet. Implicit feedback may include, by way of example, whether the clinician followed a recommended action within a defined time window; whether a suggested medication, test, referral, or diagnosis was added, modified, or removed; dwell time on a recommendation; scrolling or expansion events; repeated dismissals for similar items; overrides of alert logic; and downstream documentation or coding changes associated with the encounter. In some embodiments, each feedback event is normalized into a structured feedback record that includes: an identifier of the insight item (and optionally the underlying rule/model version that generated it), encounter and patient-context features (e.g., care setting, chief complaint, active problems, medications, recent labs/imaging), clinician role or specialty metadata, timestamps, and a feedback type and value. Feedback may further be linked to outcome proxies (e.g., later lab normalization, reduced readmission risk score, avoidance of contraindicated prescribing), where available and appropriate, to enable outcome-weighted refinement. In some embodiments, captured feedback is stored in a secure feedback repository as an append-only event stream or audit log with cryptographic integrity checks and versioning to preserve provenance. Feedback records may be encrypted at rest and in transit, access-controlled using role-based permissions, and logically partitioned to separate identifiable patient data from learning features used for model refinement.

In some embodiments, the repository enforces retention policies, patient privacy and compliance constraints, and lineage tracking such that any subsequent model update can be traced back to the specific feedback inputs, model parameters, and configuration state used to produce the update.

In some embodiments, stored feedback is used to update (i) relevance score computation (including weights, thresholds, or ranking functions), (ii) personalization parameters for a clinician, service line, or site, and/or (iii) model calibration layers that map model outputs to actionable workflow insertion policies. For example, the system may increase the priority of insights that are frequently accepted in comparable contexts and decrease the priority of insights that are repeatedly dismissed with consistent reason codes. The system may also learn clinician-or specialty-specific presentation preferences (e.g., preferred timing, verbosity, citation format, or insertion location) while maintaining clinical correctness constraints. In some embodiments, the system uses offline learning or batch updates (e.g., nightly or periodic) to incorporate aggregated feedback and may deploy updated parameters only after validation gates are satisfied.

In some embodiments, guardrails are applied to prevent unsafe, biased, or unstable adaptation. Such guardrails may include: requiring a minimum volume of feedback events before changing a parameter; bounding the magnitude and rate of adaptive updates; using robust statistics to down-weight outliers and contradictory signals; enforcing clinical safety rules that cannot be overridden by preference learning; maintaining a “do-not-suppress” class of high-severity alerts; performing drift detection and rollback if performance degrades; and requiring human governance approval for certain classes of changes (e.g., changes affecting contraindication logic or high-risk medication guidance). In some embodiments, the system stores both pre-update and post-update parameter sets and maintains an auditable change log so that model behavior is reproducible and reversible. In some embodiments, the system further prevents leakage of protected health information by de-identifying feedback features used for training, restricting cross-tenant learning, and applying differential privacy or aggregation thresholds when generating population-level updates.

In advanced embodiments, the system may integrate with medical devices, wearable monitors, and remote patient monitoring (RPM) systems to obtain additional physiologic signals, derived metrics, and device-generated events usable for patient-specific modeling and workflow guidance. By way of example, the system may receive electrocardiogram (ECG) waveforms or rhythm classifications from a wearable or implantable cardiac monitor and, upon detecting heart rhythm irregularities (e.g., atrial fibrillation burden, pauses, tachyarrhythmia episodes, or increased ectopy), generate and/or prioritize cardiology-specific recommendations, such as anticoagulation eligibility screening prompts, rate/rhythm control considerations, or referral timing suggestions. In another example, the system may receive continuous glucose monitoring (CGM) readings, insulin delivery summaries, and/or adherence indicators from an RPM platform and identify declining glucose control (e.g., increased time-above-range, rising fasting trend, recurrent nocturnal hypoglycemia) to suggest tailored endocrinology interventions, including titration discussion prompts, medication adherence checks, nutrition counseling suggestions, or earlier follow-up scheduling. In yet another example, the system may receive imaging-derived findings, metadata, or structured radiology outputs and recognize patterns in imaging that warrant earlier specialist referral (e.g., incidental pulmonary nodule progression, high-risk mammographic features, or rapidly worsening cardiac function metrics) and may insert context-appropriate guidance into the clinician workflow. In some embodiments, device and RPM inputs are treated as first-class clinical signals alongside EHR data, and the system may fuse device streams with encounter context (e.g., symptoms, medications, comorbidities, labs) to adjust relevance scoring, urgency ranking, and the timing or location of workflow insertion.

Where integration is intended to be claimed, the system may provide one or more standardized and/or proprietary interfaces for ingesting device and RPM data, normalizing the data into a common representation, and validating the data prior to use in decision support, prioritization, or model refinement. In some embodiments, interfaces include application programming interfaces (APIs) exposed by an RPM vendor or device gateway (e.g., HTTPS/REST endpoints, webhook callbacks, streaming channels, or message-queue topics), local connectivity interfaces (e.g., Bluetooth Low Energy, Wi-Fi, near-field communication, or hub-based aggregation), and/or enterprise clinical messaging interfaces (e.g., integration engines receiving HL7 v2 messages, CDA documents, or FHIR-based resources). In some embodiments, imaging-related integration may use DICOM objects and/or web-accessible imaging formats for retrieval of study metadata and derived measurements. Data formats may include structured payloads in JSON or XML, device telemetry frames, waveform encodings, and/or standardized clinical resources (e.g., observations, diagnostic reports, medication administrations, device metrics, or encounter-linked monitoring summaries), each optionally accompanied by units, sampling characteristics, timestamps, and quality indicators.

In some embodiments, the system performs multi-layer validation before incorporating integrated signals into patient-specific modeling or presenting recommendations.

In some embodiments, the multi-layer validation is performed before device-generated data or patient-reported data is incorporated into categorization of the clinical feed, patient-specific modeling, relevance score computation, adaptive weighting, thresholding, or workflow insertion decisions. In some embodiments, device-generated data includes measurements, events, telemetry, derived metrics, waveform features, or device status signals received from a medical device, wearable device, remote patient monitoring platform, or clinical equipment source. In some embodiments, patient-reported data includes symptom reports, adherence reports, questionnaire responses, self-entered measurements, caregiver-entered observations, patient portal submissions, or other patient-contributed data associated with the active patient context.

In some embodiments, validation includes data source verification to confirm that device-generated data or patient-reported data originated from an expected and permitted source. In some embodiments, data source verification includes authenticated device registration, signed payload verification, verification of a patient portal session, verification of a caregiver authorization state, or association of the data source with a patient identifier or pseudonymized active patient context identifier. In some embodiments, validation includes data format and completeness checks to determine whether required fields, timestamps, units, metadata, and expected content elements are present and interpretable prior to further use.

In some embodiments, validation includes range and consistency validation, including physiologic plausibility checks, cross-signal consistency checks, comparison to historical values for the same patient, and comparison to contemporaneous structured clinical data in the clinical feed. In some embodiments, validation includes signal quality assessment, including artifact detection, missingness thresholds, sensor wear-time confidence, signal-to-noise evaluation, dropout detection, and quality scoring for device-generated streams. In some embodiments, validation includes temporal validation, including timestamp plausibility, detection of stale or duplicate data, alignment to the active encounter or active workflow instance, and evaluation of whether the data is temporally consistent with other clinical feed updates.

In some embodiments, validation further includes error correction or standardization before incorporation into scoring. In some embodiments, error correction or standardization includes unit normalization, terminology normalization, structured mapping of free-text patient-reported content into predefined fields, deduplication, resolution of conflicting values, formatting correction, and transformation of measurements into a common representation suitable for comparison across sources. In some embodiments, patient-reported data verification includes requesting confirmation from a patient, caregiver, or clinician when a reported value is inconsistent with historical data, recent device-generated measurements, or other structured clinical data in the clinical feed.

In some embodiments, a confidence score is assigned to validated device-generated data or patient-reported data, and the confidence score is used to determine whether the data is eligible for inclusion in relevance scoring or to adjust the weight contribution of the data to one or more factor scores. In some embodiments, lower-confidence signals are down-weighted, require corroboration from another source, or are prevented from triggering high-severity or interruptive workflow insertion. In some embodiments, higher-confidence signals increase the contribution of a corresponding factor score to the relevance score when the validated signal is clinically significant for the active patient context. In some embodiments, only validated device-generated data and validated patient-reported data are incorporated into one or more factor scores used in relevance score computation, and confidence values associated with such validated data may adjust the weight contribution of the corresponding factor scores.

In some embodiments, validation outcomes, confidence values, correction operations, source identifiers, and provenance metadata are recorded in an audit trail or log repository so that the basis for incorporating or excluding device-generated data or patient-reported data from scoring is auditable and reproducible. In some embodiments, the audit trail includes an indication of validation success or failure, one or more reasons for exclusion or down-weighting, and an association with the recommendation identifier, the active patient context identifier, or both.

Validation may include: device identity verification (e.g., authenticated device registration, certificate-based trust, signed payload verification, or secure token exchange); schema validation (e.g., required fields present, type checking, unit conformity); temporal validation (e.g., timestamp plausibility, clock drift handling, duplicate detection, and alignment to encounter context); physiologic plausibility checks (e.g., range limits, rate-of-change limits, artifact detection, and cross-signal consistency); and data-quality scoring (e.g., signal-to-noise indicators, sensor wear-time confidence, or missing thresholds). In some embodiments, the system associates each ingested measurement or event with a confidence value and may gate downstream actions (e.g., suppressing low-confidence alerts, requesting confirmation, or deferring workflow insertion) based on one or more confidence thresholds. In some embodiments, the system retains provenance metadata indicating the source device/RPM system, firmware/software versions when available, ingestion pathway, and validation outcomes, thereby enabling auditability, reproducibility, and safe rollback if an integration feed is degraded or misconfigured.

In some embodiments, the system supports device enrollment, pairing, and lifecycle management for medical devices, wearables, and RPM sources. Enrollment may include associating a device identifier and/or RPM account with a patient identifier, verifying device authenticity (e.g., via signed device certificates, trusted gateways, or authenticated vendor tokens), and establishing a secure communication context for ongoing data transfer. In some embodiments, the system captures and stores patient consent and authorization artifacts prior to ingesting or using device-derived signals, including consent scope (e.g., which device streams, which care team roles, and which purposes such as monitoring, decision support, or documentation assistance), consent duration, and revocation status, and enforces such consent at ingestion time and at downstream use time. In multi-tenant deployments, the system may maintain tenant and/or site segregation such that device streams, feedback records, and derived features are logically isolated by tenant/site boundaries, with access controls and cryptographic partitioning to prevent cross-tenant data exposure and to constrain learning or aggregation to authorized scopes.

In some embodiments, the system implements fallback behavior when an integrated feed is unavailable, delayed, incomplete, or degraded. For example, the system may detect feed drop conditions using heartbeat messages, missingness thresholds, latency thresholds, or validation failures, and may (i) reduce or disable device-dependent recommendations, (ii) annotate recommendations with a data freshness indicator, (iii) request confirmation from the clinician or patient, (iv) revert to EHR-only inference paths, and/or (v) trigger a technical remediation workflow (e.g., re-pairing prompts or support tickets). In some embodiments, validated device signals influence relevance scoring and workflow insertion policies by contributing one or more features to a relevance score computation, confidence gating, and/or priority ranking. For example, measurements or events that pass validation with high confidence may increase the urgency or rank of a corresponding insight item and may cause earlier or more prominent insertion into designated workflow locations, while low-confidence or stale device signals may be down-weighted, require corroboration from other data sources, or be prevented from triggering high-severity alerts. In some embodiments, the system stores provenance and validation outcomes alongside any score contribution so that the basis for a workflow insertion decision is auditable and reproducible.

The framework may operate across inpatient, outpatient, telehealth, and home-based care environments while maintaining compliance with applicable healthcare privacy and security standards. In some embodiments, the system supports heterogeneous deployment topologies, including hospital networks, ambulatory clinics, clinician mobile devices, telehealth platforms, and home gateways used for remote monitoring, while maintaining consistent identity, authorization, and audit semantics across those environments. For example, the system may enforce environment-specific policies that account for differing threat models and connectivity constraints, such as (i) restricting high-privilege actions to managed devices when operating within an inpatient network, (ii) enforcing additional session controls or step-up authentication for telehealth access from untrusted networks, and (iii) operating in a degraded or offline-tolerant mode for home-based care where intermittent connectivity may occur. In some embodiments, the framework further maintains secure segregation between clinical tenants, sites, or affiliated entities, and may implement controlled data exchange pathways (e.g., least-privilege sharing, time-bounded access, and auditable disclosures) when coordination across care settings is clinically necessary.

The framework may be implemented to align with one or more regulatory and/or industry standards for protected health information and electronic protected health information, such as the HIPAA Privacy, Security, and Breach Notification Rules (and related HITECH requirements). In some embodiments, the framework implements technical safeguards consistent with HIPAA Security Rule requirements, including access control, audit controls, integrity protections, person/entity authentication, and transmission security. In some embodiments, the framework further maps control implementation to a structured cybersecurity control catalog or risk framework (e.g., NIST Cybersecurity Framework mappings and/or NIST security and privacy controls) to support systematic risk management and control coverage. In embodiments involving regulated electronic records and signatures (e.g., clinical investigations or other regulated workflows), the framework may additionally implement controls consistent with requirements for trustworthy and reliable electronic records (e.g., secure, time-stamped audit trails and related system controls).

In some embodiments, the technical controls include one or more of: (i) strong identity and authentication controls such as unique user identifiers, role-based and/or attribute-based access control, and multi-factor authentication; (ii) session controls such as automatic logoff and emergency “break-glass” procedures with heightened auditing; (iii) encryption of sensitive data in transit and at rest, including cryptographic key management and rotation; (iv) secure transmission channels for inter-system exchanges (e.g., TLS-protected connections), including secure medical-imaging transport where applicable; (v) tamper-evident, append-only audit logging and monitoring that records access, modification, disclosure, and administrative actions; (vi) integrity verification mechanisms to detect improper alteration of clinical data; (vii) tenant/site logical isolation, data minimization, and “minimum necessary” enforcement; (viii) consent and authorization enforcement points that gate ingestion, use, and disclosure of patient data; and (ix) incident detection and response hooks that support breach analysis and notification workflows. The system may further store provenance metadata (e.g., source system, data freshness, validation outcomes, and policy decisions) alongside outputs so that decisions affecting workflow insertion are auditable and reproducible.

The foregoing description is provided to illustrate and enable various embodiments of the disclosed subject matter. The embodiments, examples, and features described herein are not intended to be exhaustive and are not intended to limit the disclosed subject matter to the precise forms disclosed. Rather, the disclosure is intended to cover all modifications, equivalents, variations, and alternatives falling within the scope of the appended claims. The scope of the disclosed subject matter is defined by the claims and their equivalents, and nothing in this specification should be interpreted as limiting claim scope except where a feature is expressly recited in a claim.

It should be understood that the various features described throughout this specification may be combined in any manner and in any combination, except where such combinations are mutually exclusive or expressly disclaimed. Features described as part of one embodiment may be used in other embodiments, and elements described in the context of a method or process may be implemented as part of a system, apparatus, device, or computer-readable medium, and vice versa. Any described functionality may be distributed, consolidated, replicated, divided, or otherwise re-arranged across components, devices, modules, or services without departing from the scope of the disclosure.

Unless the context clearly requires otherwise, the words “comprise,” “comprising,” “include,” “including,” and “having” are intended to be construed as open-ended terms (i.e., meaning “including, but not limited to”). The term “or” is intended to be inclusive (i.e., “A or B” means A, B, or both). The terms “and/or” and “at least one of” are intended to be inclusive. In particular, the phrase “at least one of A, B, and C” is intended to mean any non-empty subset of {A, B, C} (e.g., A alone; B alone; C alone; A and B; A and C; B and C; or A and B and C). The singular forms “a,” “an,” and “the” include plural referents and vice versa.

Unless otherwise indicated, words such as “approximately,” “about,” “substantially,” and similar modifiers are intended to include ordinary tolerances, measurement error, rounding, and implementation variability consistent with the relevant technology and context. Where numerical ranges are provided, the ranges are intended to include all subranges and values within the stated ranges, including endpoints, unless expressly stated otherwise. Any examples of weights, thresholds, intervals, time windows, scaling factors, or distributions are illustrative and may be varied, re-parameterized, normalized, or transformed (including via monotonic transformations) without departing from the scope of the disclosure.

As used herein, the terms “based on,” “in response to,” “according to,” or “using” are intended to mean “based at least in part on,” “in response at least in part to,” “according at least in part to,” or “using at least in part,” unless the context clearly indicates otherwise. Accordingly, where a component, model, or logic is described as determining an output “based on” one or more inputs, the output may be determined using additional inputs, signals, or constraints not explicitly listed. Likewise, references to “determining,” “computing,” “selecting,” or “generating” include rule-based, statistical, machine learning-based, heuristic, hybrid, or other techniques, including combinations thereof.

As used herein, the term “configured to” (and similar phrases such as “adapted to,” “arranged to,” “operable to,” or “capable of”) denotes capability, structure, or programming that enables the stated function, and does not require that the function be performed at all times. References to “modules,” “engines,” “components,” “units,” “services,” or “systems” may be implemented in hardware, software, firmware, or any combination thereof, including as one or more processors executing instructions stored on one or more non-transitory computer-readable media. A described “server” may be implemented as one or more physical or virtual machines, containers, or distributed services. A described “database” may be implemented as one or more data stores, indexes, logs, queues, or other storage structures, whether centralized or distributed.

References to an element being “coupled,” “connected,” “in communication with,” or “communicatively coupled” to another element may include direct and/or indirect coupling, including coupling through one or more intermediate components, networks, buses, gateways, APIs, adapters, brokers, or other interfaces. References to “secure” channels, sessions, or controls include any one or more confidentiality, integrity, authenticity, authorization, auditability, replay protection, non-repudiation, resilience, or privacy-preserving properties as appropriate to the context, and may be implemented using any suitable mechanisms, whether now known or later developed.

Any ordering of steps, operations, or blocks described in the specification (including in flow diagrams) is provided for convenience and illustration. Unless expressly stated otherwise, steps may be performed in different orders, performed concurrently, repeated, combined, subdivided, or omitted, and still achieve desired results. Similarly, decision points, thresholds, lockout periods, gating logic, and workflow insertion modalities may be adjusted dynamically, adaptively, or contextually as described herein, without departing from the scope of the disclosure.

The headings, labels, paragraph numbers, and sectional organization used herein are for convenience only and do not limit the disclosure. Any reference numerals or figure references (if present) are provided for convenience and do not limit the claims. Descriptions of “preferred,” “example,” “illustrative,” “in some embodiments,” “in one embodiment,” or similar language indicate examples and do not imply that such embodiments are required, exclusive, or that any feature is essential unless expressly stated as such.

No feature, structure, act, or step is intended to be dedicated to the public or to constitute a disclaimer, waiver, or disavowal of claim scope unless such intent is expressly stated. The use of the terms “must,” “required,” or similar terms, if any, is not intended to imply that a feature is essential, unless expressly stated as essential. Any described advantages, benefits, or results are illustrative and may be present in some embodiments and absent in others.

Unless a claim element is expressly recited using the phrase “means for” or “step for” (or a similar formulation intended to invoke 35 U.S.C. § 112(f)), no claim element is intended to be interpreted under 35 U.S.C. § 112(f) and, instead, claim elements are intended to be construed according to their ordinary and customary meaning as understood by a person of ordinary skill in the art in view of this specification.

Accordingly, while specific embodiments have been described herein, it will be apparent to those of ordinary skill in the art that many modifications and variations may be made without departing from the broader spirit and scope of the disclosure. The appended claims are intended to cover such modifications and variations.

What is claimed is:

1. A computer-implemented method for adaptive clinical workflow management, the method comprising:
receiving, by a multi-layer adaptive clinical workflow engine, a clinical feed associated with an active patient context;
determining, based on the clinical feed, a categorization indicative of a patient state or clinical context;
generating a candidate set of replacement media for possible insertion into a clinical workflow;
computing, for each candidate replacement media item of the candidate set, a relevance score using a weighted aggregation of multiple relevance factors including at least one of the following: a patient-state factor, a treatment-context factor, a provider-related factor, an operational-resource factor, a geographic factor or an epidemiological factor;
comparing the relevance score to a relevance threshold;
selecting a candidate replacement media item for workflow insertion only when the relevance score satisfies the relevance threshold; and
overriding the clinical feed with the selected replacement media by inserting contextualized guidance into the clinical workflow.

2. The method of claim 1, wherein the operational-resource factor is based on one or more of pharmaceutical inventory data, device availability data, or care-team capacity data.

3. The method of claim 1, wherein the operational-resource factor is based on stock levels of pharmaceuticals or locations of medicines, and wherein, when a preferred medication is unavailable, the method further comprises decreasing a relevance score for replacement media associated with the preferred medication and selecting replacement media suggesting an alternative medication that is clinically indicated and operationally available.

4. The method of claim 1, wherein the replacement media includes a suggestion of a treatment, product, or pharmaceutical based on at least one of a recent regulatory approval of a product, treatment, or pharmaceutical, updated clinical guidelines, updated reference information, or more general medical information received in the clinical feed or a remote clinical knowledge database.

5. The method of claim 1, further comprising, during the adaptively calibrating, adjusting the weighting factor or the relevance threshold subject to safeguards including at least one of: bounding a magnitude of change within a predetermined period, requiring a minimum amount of clinician interaction data before adjustment, maintaining a baseline configuration, or reverting to the baseline configuration when increased alert fatigue or degraded performance is detected.

6. The method of claim 1, further comprising implementing a learning loop that logs clinician interaction events including acceptance, modification, deferral, feedback, ignoring, or override events, links the clinician interaction events to a recommendation identifier and an active patient context identifier, and uses the linked clinician interaction events together with outcome indicators derived from subsequent updates to the clinical feed to refine future relevance score computation.

7. The method of claim 1, further comprising, during the validating, performing one or more of:
data-source verification,
data-format and completeness checking,
range or consistency validation,
signal-quality assessment,
temporal validation,
error correction or standardization, or
provenance logging.

8. The method of claim 1, further comprising receiving dictated clinical documentation from a physician, nurse, or other medical personnel, performing speech-to-text transcription and normalization of the dictated clinical documentation, and generating editable documentation suggestions for insertion into patient notes based at least in part on the clinical feed.

9. The method of claim 1, wherein the replacement media is generated from a remote clinical knowledge database containing evidence-based guidelines, clinical trial data, real-world outcomes data, predictive models, or pre-validated clinical explanations, and wherein the replacement media includes patient-specific values inserted into a pre-validated clinical explanation.

10. The method of claim 1, further comprising caching, at a physician device, selected replacement media or a preformatted presentation payload for rapid insertion during a workflow transition, and invalidating cached content based on at least one of encounter termination, change to the clinical feed, change to medication-interaction risk, change to patient state assessment, or change in applicable time-sensitive guidance.

11. A system for adaptive clinical workflow management, comprising:
one or more processors; and
one or more non-transitory computer-readable media storing instructions that, when executed by the one or more processors, cause the system to implement a multi-layer adaptive clinical workflow engine configured to:
receive a clinical feed associated with an active patient context;
determine, based on the clinical feed, a categorization indicative of a patient state or clinical context;
generate a candidate set of replacement media for possible insertion into a clinical workflow;
compute, for each candidate replacement media item of the candidate set, a relevance score using a weighted aggregation of multiple relevance factors including at least one of the following: a patient-state factor, a treatment-context factor, a provider-related factor, an operational-resource factor, a geographic factor, or an epidemiological factor;
compare the relevance score to a relevance threshold;
select a candidate replacement media item for workflow insertion only when the relevance score satisfies the relevance threshold; and
override the clinical feed with the selected replacement media by inserting contextualized guidance into the clinical workflow.

12. The system of claim 11, wherein the operational-resource factor is based on one or more of pharmaceutical inventory data, device availability data, or care-team capacity data.

13. The system of claim 11, wherein the operational-resource factor is based on stock levels of pharmaceuticals or locations of medicines, and wherein, when a preferred medication is unavailable, the system is configured to decrease a relevance score for replacement media associated with the preferred medication and to select replacement media suggesting an alternative medication that is clinically indicated and operationally available.

14. The system of claim 11, wherein the replacement media includes a suggestion of a treatment, product, or pharmaceutical based on at least one of a recent regulatory approval of a product, treatment, or pharmaceutical, updated clinical guidelines, updated reference information, or more general medical information received in the clinical feed or a remote clinical knowledge database.

15. The system of claim 11, further comprising instructions that, when executed, cause the multi-layer adaptive clinical workflow engine, during the adaptive calibration, to adjust the weighting factor or the relevance threshold subject to safeguards including at least one of: bounding a magnitude of change within a predetermined period, requiring a minimum amount of clinician interaction data before adjustment, maintaining a baseline configuration, or reverting to the baseline configuration when increased alert fatigue or degraded performance is detected.

16. The system of claim 11, wherein the multi-layer adaptive clinical workflow engine is further configured to:
log clinician interaction events including acceptance, modification, deferral, feedback, ignoring, or override events,
link the clinician interaction events to a recommendation identifier and an active patient context identifier, and
refine future relevance score computation using the linked clinician interaction events together with outcome indicators derived from subsequent updates to the clinical feed.

17. The system of claim 11, further comprising instructions that, when executed, cause the multi-layer adaptive clinical workflow engine, during the validation, to perform one or more of:
data-source verification,
data-format and completeness checking,
range or consistency validation,
signal-quality assessment,
temporal validation,
error correction or standardization, or
provenance logging.

18. The system of claim 11, wherein the multi-layer adaptive clinical workflow engine is further configured to receive dictated clinical documentation from a physician, nurse, or other medical personnel, perform speech-to-text transcription and normalization of the dictated clinical documentation, and generate editable documentation suggestions for insertion into patient notes based at least in part on the clinical feed.

19. The system of claim 11, further comprising a remote clinical knowledge database containing evidence-based guidelines, clinical trial data, real-world outcomes data, predictive models, or pre-validated clinical explanations, wherein the multi-layer adaptive clinical workflow engine is configured to select replacement media from the remote clinical knowledge database and to populate patient-specific values into a pre-validated clinical explanation.

20. The system of claim 11, wherein the multi-layer adaptive clinical workflow engine is further configured to cache selected replacement media or a preformatted presentation payload for rapid insertion during a workflow transition and to invalidate cached content based on at least one of encounter termination, change to the clinical feed, change to medication-interaction risk, change to patient state assessment, or change in applicable time-sensitive guidance.
